(19) United States
(12) Patent Application Publication
(10) Pub. No.: US 2009/0317413 A1
(43) Pub. Date: Dec. 24, 2009

(54) DETECTION OF HUMAN PLATELET ANTIGEN (HPA) ANTIBODIES

(75) Inventors: Prachi Stafford, Cambridge (GB); Nicholas Watkins, Cambridge (GB); Willem Ouwehand, Cambridge (GB)

Correspondence Address: BANNER & WITCOFF, LTD., 28 STATE STREET, SUITE 1800, BOSTON, MA, 02109-1701, US

(73) Assignee: Cambridge Enterprise Limited, Cambridge (GB)

(21) Appl. No.: 12/121,059

(22) Filed: May 15, 2008

Related U.S. Application Data
(63) Continuation of application No. PCT/GB2006/004262, filed on Nov. 15, 2006.

(30) Foreign Application Priority Data
Nov. 15, 2005 (GB) 0523226.5
Jun. 14, 2006 (GB) 0611806.1

(51) Int. Cl. A61K 39/395; C07K 14/435; C12N 15/11; C12N 15/00; C12N 5/00; G01N 33/53; C12P 21/00; A61P 43/00

(52) U.S. Cl. 424/185.1; 530/324; 530/350; 536/23.5; 435/320.1; 435/348; 435/7.21; 435/71.1; 436/501

(57) ABSTRACT

This invention relates to recombinant GPIIIa molecules that lack the ligand binding βA domain and bind to HPA-1 antibodies in monomeric form. These GPIIIa molecules can be recombinantly expressed at high levels and may be useful, for example, in the detection of HPA-1 antibodies. Such detection may be useful, for example, in the diagnosis and therapy of HPA antibody disorders such as Neonatal Alloimmune Immune Thrombocytpenia (NAIT), Post Transfusion Purpura (PTP) and Platelet Refractoriness (PR).

### RELATED APPLICATIONS

This application is a continuation of PCT application no. PCT/GB2006/004262, designating the United States and filed Nov. 15, 2006; which claims the benefit of the filing dates of United Kingdom application no. GB 0611806.1, filed Jun. 14, 2006; and United Kingdom application no. GB 0523266.5, filed Nov. 15, 2005; and each of which is hereby incorporated herein by reference in its entirety for all purposes.

### FIELD

This invention relates to the detection of Human Platelet Antigen (HPA) antibodies, in particular human alloantibodies reactive with integrin GPIIIa. Such detection may be useful, for example, in the diagnosis and therapy of HPA antibody disorders such as Neonatal Alloimmune Immune Thrombocytpenia (NAIT), Post Transfusion Purpura (PTP) and Platelet Refractoriness (PR).

### BACKGROUND

Antibodies against Human Platelet Antigens (HPA) are involved in three clinical syndromes of Neonatal Alloimmune Immune Thrombocytpenia (NAIT), Post Transfusion Purpura (PTP) and Platelet Refractoriness (PR). In the Caucasoid population, HPA-1a (previously known as Zw^{a}/P1^{A})¹ on platelet glycoprotein GPIIIa (CD61, β3) is the clinically most important HPA antigen. The HPA-1a/1b dimorphism is based on a thymidine (t) for cytosine (c) Single Nucleotide Polymorphism (SNP) at position 176 of the ITGB3 gene (NCBI SNP database entry rs5918, see Worldwide Web site: ncbi.nlm.nih.gov/entrez/query.fcgi?db=snp, resulting in a leucine (HPA-1a) to proline (HPA-1b) substitution at position 33 of the mature protein^{2,3}. The HPA-1 system is the clinically most relevant HPA system. Alloantibody formation is generally against HPA-1a. HPA-1b antibodies are rare and are mainly observed in polytransfused patients and occasionally observed in pregnancy. Approximately 2-2.5% of Caucasoids are HPA-1b1b. In pregnancy HPA-1b1b women (HPA-1a negative) are at high risk (with a 1 in 3 chance) of forming HPA-1a antibodies if HLA DRB3*0101 positive. HPA-1a antibody formation in HLA DRB3*0101 negative pregnant women is very rare (odds ratio of ˜150 when compared with HLA DRB3*0101 positive women).

Several prospective screening studies in pregnant women have shown that 1 in 365 (1 in 9 of the HPA-1b1b ones) women form HPA-1a antibodies and severe thrombocytopenia in the fetus/neonate occurs in 1 in 1200 cases⁴⁻⁶. Intra-cerebral haemorrhage (ICH) and other major bleeds only present in a small subset of cases with severe thrombocytopenia. There are currently no reliable laboratory tests to predict the risk of severe thrombocytopenia and of severe bleeding in fetuses/neonates born to mothers with HPA-1a alloantibodies. The precise incidence of severe thrombocytopenia with ICH is not known but it is thought to be as high as 1 in 10-20 000 term pregnancies (30-60 cases per annum in the UK). ICH occurs during or after delivery but may also occur in utero as early as 20-24 weeks of gestation^{7,8}. The availability of a simple diagnostic test which could differentiate with good sensitivity and reasonable specificity between HPA-1a antibodies which cause severe bleeding and possible ICH and the less harmful HPA-1a antibodies would be of great clinical value.

Currently, there is no routine antenatal screening for HPA-1a antibodies because cases at risk of severe bleeding and/or ICH cannot be reliably identified within the group of women with HPA-1a antibodies. Therefore, investigations are generally only carried out in response to a clinical referral from a neonate with an isolated and low platelet count, if there is a history of HPA-1a alloimmunization or if a low platelet count in the neonate has been identified coincidentally because a full blood count was performed.

Detection of HPA antibodies is based on solid phase based antigen-capture assays like the Monoclonal Antibody Immobilization of Platelet Antigens (MAIPA) assay, the indirect Platelet Suspension Immunofluorescence Test (PSIFT, either read by microscopy or by FACS) or commercial ELISA kits in which the wells of the ELISA plates are coated with platelet membrane glycoprotein complexes obtained from human HPA typed platelets (e.g. GTI-Pak12, GTI, Milwaukee, Wis., USA). These methods are widely used, but have major drawbacks. The MAIPA is cumbersome and time-consuming and requires panels of HPA genotyped donor platelets. The PSIFT is laborious and not specific (HLA antibodies also produce positive results). GTI-Pak has a slightly lower sensitivity than either MAIPA or PSIFT.

Several groups have been attempting to develop recombinant or synthetic molecular probes that can be used for the sensitive and specific detection of HPA antibodies in general and HPA-1 antibodies in particular. The latter studies have been hampered by the complexity of the integrin structure in general and of the HPA-1 epitopes in particular. Many studies have tried to elucidate the exact nature of the HPA-1 epitopes and the precise structural configuration of the HPA-1 antibody binding sites, but success has been limited so far.

It has been shown that the tertiary structure of GPIIIa, and in particular that of the amino terminal domains, is critical for the binding of many HPA-1 antibodies⁹ as HPA-1 antibodies do not bind overlapping linear 13-mer polypeptides straddling residue 33 (Flug et al. (1991) Blood 77(9):1964-9).

Some examples of HPA-1 antibodies bind to a GPIIIa fragment consisting of the amino-terminal 66 amino acids, when recombinantly expressed in Escherichia coli (Bowditch et al. Blood Feb. 1, 1992, 79(3) :559-62), but this fragment is not adequate for HPA-1 antibody detection as many examples of clinically relevant HPA-1a antibodies do not bind. Further studies using site-directed mutagenesis of the amino terminal cysteines in GPIIIa have shown the importance of the long range disulfide bond between cysteine 5 (now remapped to cysteine 13) and 435, as the replacement of cysteine 435 by alanine was detrimental to the binding of so called category II type HPA-1a antibodies (Valentin et al. Blood, Jun. 1, 1995, 85(11):3028-3033).

Although GPIIIa has been expressed as a soluble monomer by deleting the specificity-determining loop¹⁶, there is no indication that the HPA-1 epitope is conserved in such monomers.

More recently, we have observed the importance of residue 93 in the GPIIIa hybrid domain for maintaining the integrity of the HPA-1a epitope since the replacement of glutamine-93 by glycine resulted in the loss of binding of all three known examples of recombinant human HPA-1a antibodies (Watkins et al. Blood, Mar. 1, 2002, 99(5):1833-1839). Finally, we have observed that even a minor modification of residue 33 from leucine to valine (which is the result of a very rare GPIIIa sequence haplotype) has a detrimental effect on the binding of our recombinant HPA-1a antibody CamTran007 but not of two other recombinant human HPA-1a antibodies, clones 19.7 and 23.15. Thus several studies have confirmed that for adequate HPA-1 antibody binding GPIIIa residues outside the plexin-semaphorin-integrin (PSI) domain are critical.

### SUMMARY

The present inventors have recognised that recombinant GPIIIa molecules that lack the ligand binding βA domain bind to HPA-1 antibodies in monomeric form and can be recombinantly expressed at high levels.

One aspect of the invention provides a GPIIIa polypeptide comprising an amino acid sequence which has at least 80% sequence identity or sequence similarity with the sequence of SEQ ID NO: 1 from residue 1 to at least residue 54.

In certain exemplary embodiments, the GPIIIa polypeptide comprises an amino acid sequence which has at least 80% sequence identity or sequence similarity with the sequence of SEQ ID NO: 1 from residue 1 to at least residue 104, at least residue 192, at least residue 289, at least residue 322, at least residue 365, or residue 450.

In certain exemplary embodiments, the GPIIIa polypeptide comprises an amino acid sequence which shares greater than 85%, greater than 90%, greater than 95% or greater than 98% sequence identity or sequence similarity with such a sequence.

For example, the polypeptide may have a sequence with one or more of addition, insertion, deletion or substitution of one or more amino acids from the corresponding sequence in SEQ ID NO: 1. For example, up to about 5, 10, 15, 20, 30 or 40 amino acids may be altered. Such alterations may be caused by one or more of addition, insertion, deletion or substitution of one or more nucleotides in the encoding nucleic acid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 shows a schematic diagram of the domains of GPIIIa employed in the constructs described herein.

FIG. 2 shows a schematic diagram of the cloning of GPIIIaPSIHyb fragment and GPIIIaΔβA in puC119-MH.

FIG. 3 shows the results of nucleotide sequencing of the gene constructs used for the generation of the GPIIIaΔSDL-P33 (lower panel) which was derived from the GPIIIaΔSDL-L33 (upper panel) by site-directed mutagenesis.

FIG. 4 shows the expression of recombinant GPIIIaΔSDL in CHO Lec 3.2.8.1 cell supernatant. Bound murine monoclonal IgG (Y2/51, a monoclonal antibody specific for GPIIIa in the absence of GPIIb; 9E10, a isotype matched “negative control” monoclonal antibody against the Myc antigen) was detected with horseradish peroxidase-conjugated goat anti-mouse antibody and absorbance read at 450 nm. Each data point represents an average of three replicate samples.

FIG. 5 shows the reactivity of GPIIIaΔSDL-L33 and GPIIIaΔSDL-P33 with a panel of monoclonal antibodies by ELISA (monoclonal antibody Y2/51 as per FIG. 4; monoclonal antibody RFGP56 against GPIIbIIIa and known not to react with GPIIIa in the absence of GPIIb; monoclonal antibody 9E10 as per FIG. 4). Absorbance was read at 450 nm; Sup—supernatant; Lys—Lysate. Each OD represents the mean of duplicate determinations.

FIG. 6 shows ELISA analysis of the binding of secreted GPIIIaΔSDL-L33 (black) or GPIIIaΔSDL-P33 (grey) to a panel of human recombinant HPA-1a antibodies (Antibodies CamTran007, 19.7 and 23.15 are recombinant human phage antibodies [re-engineered to human IgG] and specific for HPA-1a; antibody Fog is against an irrelevant antigen, RhD). Absorbance was read at 450 nm; X-axis, monoclonal antibodies; Y-axis, absorbance value. Each OD represents the mean of duplicate determinations.

FIG. 7 shows ELISA analysis of the binding of secreted GPIIIaΔSDL-L33 (black) or GPIIIaΔSDL-P33 (grey) to three polyclonal human sera (diluted 1:20; anti-HPA-1a contains alloantibodies against HPA-1a; anti-HPA-1b, contains alloantibodies against HPA-1b; AB, inert serum from a donor with blood group AB). Absorbance was read at 450 nm; X-axis, human samples with polyclonal antibodies; Y-axis, absorbance value. Each OD represents the mean of duplicate determinations.

FIG. 8 shows ELISA analysis of the binding of secreted GPIIIa fragments (PSI-A33L33, PHyb-L33 [=PSIHyb-L33 in other figures], ΔβA-L33 ΔSDL-L33; ΔSDL-P33 and the two Ig folds of human glycoprotein VI [D1D2] as negative controls)to 18 human plasma or serum samples (labelled A-R) containing IgG HPA-1a antibodies from cases of neonatal alloimmune thrombocytopenia (NAIT). In each of the three panels recombinant HPA-1a antibody CamTran0007 has been used as a positive control). This antibody does not react with the two shortest fragments (PSI-A13L33 and PHyb-L33).

FIG. 9 shows the reactivity of GPIIIa calmodulin tagged fragments with GPIIIa MAbs.

FIGS. 10A and 10B depict the reactivity of murine GPIIIa specific monoclonal antibodies with recombinant GPIIIa in ELISA: (A) shows the binding of three mouse GPIIIa antibodies CRC54, Y2/51 and RFGP56 (10 μg/mL) with the recombinant GPIIIa in ELISA; and (B) shows binding of the mouse monoclonal antibody SZ21 at two concentrations 1 μg/mL and 10 μg/mL with recombinant GPIIIa fragments.

FIG. 11 shows the reactivity of the human HPA-1a 19-7, 23-15 and CamTran007 to recombinant GPIIIa fragments in ELISA.

FIGS. 12A and 12B depict antibody reactivity: (A) shows the reactivity of the WHO anti-HPA-1a minimum potency standard (NIBSC 93/710) with recombinant GPIIIa fragments ΔβA and ΔSDL by ELISA; and (B) shows the reactivity of the WHO proposed anti-HPA-1a sensitivity standard (NIBSC 03/152) with recombinant GPIIIa fragments ΔβA and ΔSDL by ELISA. The level of detection achieved in the MAIPA is shown by the dotted lines in both panels. Points represent the means±SD of triplicate wells in one experiment, representative at least two experiments.

FIG. 13 shows the reactivity of HPA-1b antibodies with the recombinant GPIIIa fragments. Five HPA-1b antibodies were tested for reactivity with the recombinant calmodulin-tagged GPIIIa fragments by ELISA. As positive control, reactivity of the leucine 33 form of ΔSDL with the anti-HPA-1a CamTran007 was included and as negative control, all anti-HPA-1bs were also tested for reactivity with recombinant GPVI (D1D2).

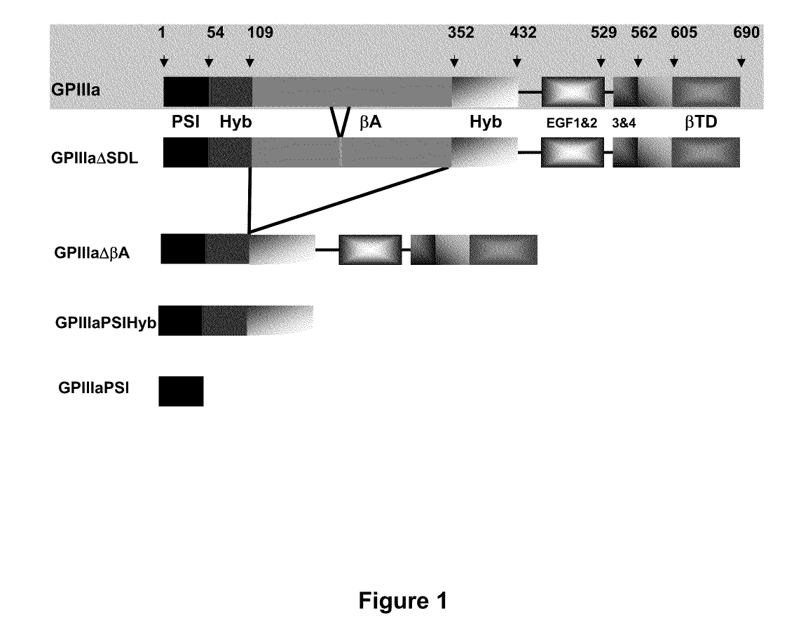

### DETAILED DESCRIPTION

Sequence similarity and identity are commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm (Nucl. Acids Res. (1997) 25:3389-3402) may be used.

Sequence identity and similarity may also be determined using Genomequest™ software (Gene-IT, Worcester Mass. USA). Sequence comparisons are typically made over the entire length of the relevant sequence described herein.

Similarity allows for “conservative variation,” i.e. substitution of one hydrophobic residue such as isoleucine, valine, leucine or methionine for another, or the substitution of one polar residue for another, such as arginine for lysine, glutamic for aspartic acid, or glutamine for asparagine. Similarity matrices such as the PAM 250 or Blosum 45 are commonly used in the art to determine similarity and conservative variation.

In certain exemplary embodiments, the GPIIIa polypeptide may comprise the amino acid sequence of SEQ ID NO: 1 from residue 1 to at least residue 54, at least residue 108, at least residue 192, at least residue 289, at least residue 322, at least residue 365 or residue 450 For example, the GPIIIa polypeptide may comprise residues 1 to 54 of SEQ ID NO: 1, residues 1 to 108 of SEQ ID NO: 1, residues 1 to 192 of SEQ ID NO: 1, residues 1 to 289 of SEQ ID NO: 1, residues 1 to 322 of SEQ ID NO: 1, residues 1 to 365 of SEQ ID NO: 1 or in certain embodiments, the entire sequence of SEQ ID NO: 1 (residues 1 to 450).

A GPIIIa polypeptide as described herein binds HPA-1 antibodies. HPA-1 antibodies bind to a conformational epitope of GPIIIa which comprises the PSI domain. HPA-1 antibodies may be specific for the allele of GPIIIa which has leucine (L) at position 33 in the mature GPIIIa sequence (HPA-1a) or the allele of GPIIIa which has proline (P) at position 33 (HPA-1b). The amino acid sequence of GPIIIa has the database entry P05106 (P5106.1 GI: 124968).

A GPIIIa polypeptide as described herein may have a leucine (L) at residue 33 and may bind preferentially to HPA-1a antibodies relative to HPA-1b antibodies. In certain exemplary embodiments, such a GPIIIa polypeptide binds specifically to HPA-1a antibodies i.e., it binds to HPA-1a antibodies but shows no binding or substantially no binding to HPA-1b antibodies.

In other embodiments, the GPIIIa polypeptide may have a proline (P) at residue 33 and may bind preferentially to HPA-1b antibodies relative to HPA-1a antibodies. In certain exemplary embodiments, the GPIIIa polypeptide specifically binds to HPA-1b antibodies i.e., it binds to HPA-1b antibodies but shows no binding or substantially no binding to HPA-1a antibodies.

A GPIIIa polypeptide as described herein may also bind other HPA antibodies. For example, a GPIIIa polypeptide in which Arginine 62 is replaced by Glutamine may be used to bind HPA-10bw antibodies, a GPIIIa polypeptide in which Proline 166 of SEQ ID NO: 1 (P407 of wild-type GPIIIa) is replaced by Alanine may be used to bind HPA-7bw antibodies, a GPIIIa polypeptide in which Arginine 248 of SEQ ID NO:1 (R489 of wild-type GPIIIa) is replaced by Glutamine may be used to detect for HPA-6bw antibodies, a GPIIIa polypeptide in which Arginine 392 of SEQ ID NO: 1 (R633 of wild-type GPIIIa) is replaced by histidine may be used to detect HPA-11bw antibodies, a GPIIIa polypeptide in which Lysine 370 of SEQ ID NO: 1 (K611 of wild-type GPIIIa) is deleted may be used to detect HPA-14bw antibodies and a GPIIIa polypeptide in which Arginine 395 of SEQ ID NO: 1 (R636 of wild-type GPIIIa) is replaced by Cysteine may be used to detect HPA-8bw antibodies (for source data, see the EMBL EBI IPD-HPA database; Worldwide Web site: ebi.ac.uk/ipd/hpa).

In some embodiments, a GPIIIa polypeptide may comprise two or more substitutions or deletions as set out above in order to bind to more than one type of HPA antibody.

In some embodiments, one or more heterologous amino acids may be joined or fused to a polypeptide set out herein and a polypeptide may comprise a polypeptide sequence as described above linked or fused to one or more heterologous amino acids. One or more heterologous amino acids may include non-GPIIIa amino acid sequences.

A GPIIIa polypeptide may comprise a tag, which may, for example, be an affinity tag which is useful for purification. An affinity tag is a heterologous polypeptide sequence which forms one member of a specific binding pair. For example, the tag sequence may form an epitope which is bound by an antibody molecule. Polypeptides comprising the tag may be purified though binding to the other member of the specific binding pair, which may be immobilized, for example on an affinity column.

Suitable affinity tags include for example, glutathione-S-transferase, (GST), calmodulin, maltose binding domain (MBD), MRGS(H)₆ (SEQ ID NO:3), DYKDDDDK (SEQ ID NO:4) (FLAG™), T7-, S-(KETAAAKFERQHMDS) (SEQ ID NO:5), poly-Arg (R₅₋₆), poly-His (H₂₋₁₀), poly-Cys (C₄) poly-Phe(F₁₁) poly-Asp(D₅₋₁₆), Strept-tag II (WSHPQFEK) (SEQ ID NO:6), c-myc (EQKLISEEDL) (SEQ ID NO:7), Influenza-HA tag (Murray, P. J. et al. (1995) Anal. Biochem. 229:170-179), Glu-Glu-Phe tag (Stammers, D. K. et al. (1991) FEBS Lett. 283:298-302), Tag. 100 (Qiagen; 12 aa tag derived from mammalian MAP kinase 2), Cruz tag 09™ (MKAEFRRQESDR (SEQ ID NO:8), Santa Cruz Biotechnology Inc.) and Cruz tag 22™ (MRDALDRLDRLA (SEQ ID NO:9), Santa Cruz Biotechnology Inc.). Known tag sequences are reviewed in Terpe (2003) Appl. Microbiol. Biotechnol. 60:523-533.

In certain exemplary embodiments, a calmodulin tag may be used. The sequence of calmodulin has the database accession number BC072232.1 GI:47937654.

In some embodiments, it may be desirable to remove the affinity tag after purification. The GPIIIa polypeptide may, for example, comprise one or more protease recognition sites. A protease recognition site may, for example, be positioned between the GPIIIa sequence and the affinity tag such that contacting the polypeptide with a site-specific protease cleaves the polypeptide at the recognition site to release the GPIIIa sequence from the tag.

Various suitable protease recognition sites are known in the art, including Factor Xa, thrombin, rennin, TEV protease and enterokinase recognition sites. In certain exemplary embodiments, the protease recognition site is a tobacco etch virus (TEV) protease recognition site have the sequence Glu-X-X-Tyr-X-Gln/Ser (SEQ ID NO:10). Other suitable protease recognition sites are described in Richter et al. J. Biol. Chem. (2002) 277:43888-43894.

In some embodiments, the GPIIIa polypeptide may be coupled to an appropriate signal leader peptide to direct secretion of the GPIIIa polypeptide from cell into the culture medium. The signal leader sequence may be removed following expression to produce the mature GPIIIa polypeptide. A range of suitable signal leader peptides are known in the art.

For some applications, the GPIIIa polypeptide may be immobilized, for example, by attachment to an insoluble support. The support may be in particulate or solid form and may include a plate, test tube, bead, ball, filter or membrane. A polypeptide may, for example, be fixed to an insoluble support that is suitable for use in affinity chromatography. Methods for fixing polypeptides to insoluble supports are known to those skilled in the art.

The invention also encompasses nucleic acids, vectors and cells which are suitable for use in methods of producing a GPIIIa polypeptide, as described above.

Accordingly, a nucleic acid may comprise a nucleic acid sequence which encodes a GPIIIa polypeptide as described above. A suitable sequence may comprise the nucleic acid sequence of SEQ ID NO:2 or a fragment of SEQ ID NO:2 from base 1 to at least base 162, at least base 324, at least base 576, at least base 867, at least base 966 or at least base 1095, or a variant of any of these.

A nucleic acid sequence which is a variant of a nucleic acid sequence from SEQ ID NO: 2 as described above may share greater than 60% sequence identity, greater than 65%, greater than 70%, greater than about 80%, greater than 90% or greater than 95% with the nucleic acid sequence from SEQ ID NO: 2.

A variant may have one or more of addition, insertion, deletion or substitution of one or more nucleotides in the sequence of a nucleic acid sequence from SEQ ID NO: 2. Up to 10, 20, 30, 50 or 100 nucleotides may be added, inserted, deleted or substituted as described.

The nucleotide sequence encoding the GPIIIa polypeptide may be operably linked to a heterologous regulatory sequence. Suitable regulatory sequences to drive the expression of heterologous nucleic acid coding sequences in expression systems are well known in the art.

A heterologous regulatory sequence may be an inducible promoter. Such a promoter may induce expression in response to a stimulus. This allows control of expression, for example, to allow optimal cell growth before recombinant polypeptide production is induced. Many examples of inducible promoters are known to those skilled in the art, including the Drosophila metallothionein (MT) promoter, which is inducible with copper sulphate (CuSO₄).

The heterologous regulatory sequence may be activated by a heterologous transcription factor, such as GAL4 or T7 polymerase. For example, a GAL4 transcription factor may be expressed using an inducible promoter and may drive expression of a coding sequence which is operably linked to the GAL4 promoter. In other embodiments, T7 polymerase may be expressed using an inducible promoter and may drive expression of a coding sequence which is operably linked to a T7 promoter.

The term “heterologous” indicates that the gene/sequence of nucleotides in question or a sequence regulating the gene/sequence in question, is a recombinant sequence which has been introduced into a construct, vector or cell, artificially, using genetic engineering or recombinant means, i.e. by human intervention. Heterologous nucleotide sequences are sequences which do not naturally occur together in nature. Nucleotide sequences which are heterologous to a cell may be non-naturally occurring in cells of that type, variety or species (i.e. exogenous or foreign) or may be sequences which are non-naturally occurring in that sub-cellular or genomic environment of the cells or may be sequences which are non-naturally regulated in the cells i.e. operably linked to a non-natural regulatory element.

Nucleic acid sequences and constructs as described above may be comprised within a vector. Those skilled in the art are well able to construct vectors and design protocols for recombinant gene expression, for example in a microbial or host cell. Suitable vectors can be chosen or constructed, containing appropriate regulatory sequences, including promoter sequences, terminator fragments, polyadenylation sequences, enhancer sequences, marker genes and other sequences as appropriate. A vector may comprise a selectable marker to facilitate selection of the transgenes under an appropriate promoter. For further details see, for example, Molecular Cloning: a Laboratory Manual: 3rd edition, Sambrook & Russell, 2001, Cold Spring Harbor Laboratory Press.

Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second Edition, Ausubel et al. eds. John Wiley & Sons, 1992.

A nucleic acid construct or vector may be introduced into a host cell. When introducing or incorporating a nucleic acid construct into a cell, certain considerations must be taken into account, well known to those skilled in the art. The nucleic acid to be inserted should be assembled within a construct or vector which contains effective regulatory elements which will drive transcription. There must be available a method of transporting the constructor vector into the cell. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

The particular choice of a transformation technology will be determined by its efficiency to transform the particular host cells employed as well as the experience and preference of the operator with a particular methodology of choice.

Marker genes such as antibiotic resistance or sensitivity genes may be used in identifying cells containing the nucleic acid of interest, as is well known in the art.

A method of producing a GPIIIa polypeptide as described herein may comprise introducing a nucleic acid encoding a GPIIIa polypeptide as described above into a host cell, and expressing said nucleic acid to produce the GPIIIa polypeptide.

A nucleic acid may be expressed by culturing the host cells (which may include cells actually transformed, although more likely the cells will be descendants of the transformed cells) under conditions for expression of the nucleic acid, so that the encoded GPIIIa polypeptide is produced.

The GPIIIa polypeptide may be isolated and/or purified from the host cell.

In some embodiments, the GPIIIa polypeptide may be purified using an affinity tag, which may, optionally, be removed after purification, for example using a specific protease.

Further aspects of the invention provide a host cell comprising a vector or nucleic acid construct as described herein, and a method of producing a host cell as described herein may comprise introducing a nucleic acid as described herein into the host cell.

A host cell may contain a nucleic acid sequence encoding a GPIIIa polypeptide as a result of the introduction of the nucleic acid sequence into an ancestor cell.

A range of host cells suitable for the production of GPIIIa polypeptides are known in the art. Suitable host cells may include prokaryotic cells, in particular bacteria such as E. coli, and eukaryotic cells, including mammalian cells such as CHO and CHO-derived cell lines (Lec cells), HeLa, COS, and HEK293 cells, amphibian cells such as Xenopus oocytes, insect cells such as Trichoplusia ni, S2, Sf9 and Sf21 and yeast cells.

Other aspects of the invention relate to the detection of HPA antibodies using the GPIIIa polypeptides described herein.

A method of detecting an HPA antibody in a sample obtained from an individual may comprise determining the binding of one or more antibodies in the sample to a GPIIIa polypeptide as described herein, wherein the binding of one or more antibodies to the polypeptide is indicative of the presence of an HPA antibody in the sample. The presence of an HPA antibody in a sample obtained from an individual may be indicative that the individual has a condition associated with HPA antibodies, such as Neonatal Alloimmune Immune Thrombocytpenia (NAIT), Post Transfusion Purpura (PTP) or Platelet Refractoriness (PR) or is a blood donor. The sample may be a blood, serum or plasma sample. In some embodiments, the sample may be obtained from a pregnant female.

An HPA antibody is an alloantibody which is reactive in vivo with a human platelet antigen (HPA). In certain exemplary embodiments, the HPA antibody may be an HPA-1 antibody, for example an HPA-1a, an HPA-1b antibody or an HPA antibody against another HPA which is located on the GPIIIa polypeptide outside the βA domain, such as HPA-6w, HPA-7w, HPA-8w, HPA-10w, HPA-11w or HPA-14bw.

For example, a GPIIIa polypeptide that has a leucine (L) residue at position 33 may specifically bind to HPA-1a antibodies. A method of detecting HPA-1a antibodies in a sample obtained from an individual may comprise determining the binding of one or more antibodies in the sample to a GPIIIa polypeptide comprising L at residue 33 as described herein, wherein binding to the GPIIIa polypeptide is indicative of the presence of HPA-1a antibodies in the sample. The presence of one or more antibodies which bind to an L33 GPIIIa polypeptide which lacks EGF domains, for example a polypeptide consisting of the sequence of residues 1 to 54 of SEQ ID NO: 1 (e.g. GPIIIaPSI-A13L33) or the sequence of residues 1 to 192 of SEQ ID NO: 1 (e.g. GPIIIaPSIHyb-L33) may be indicative of an increased risk of severe bleeding and/or an increased risk of cerebral bleeds in the individual.

The presence of one or more antibodies which bind to an L33 GPIIIa polypeptide which comprises EGF domains, for example GPIIIa-ΔβA-L33 (residues 1 to 450 of SEQ ID NO:1) but do not bind to L33 GPIIIa polypeptides which lack EGF domains, may be indicative of HPA-1a antibodies which are not associated with a increased risk of severe bleeding or cerebral bleeding.

A method of detecting HPA-1a antibodies in a sample obtained from an individual may comprise determining the binding of one or more antibodies in the sample to a first GPIIIa polypeptide comprising L at residue 33 and a second GPIIIa polypeptide comprising L at residue 33, wherein the first GPIIIa polypeptide does not comprise EGF domains and the second first GPIIIa polypeptide comprises EGF domains, wherein binding to the first GPIIIa polypeptide but not the second GPIIIa polypeptide is indicative of an increased risk of severe bleeding and/or cerebral bleeds in the individual. Suitable first GPIIIa polypeptides may include polypeptides consisting of residues 1 to 54 or residues 1 to 192 of SEQ ID NO:1 as described herein (e.g. GPIIIaPSI-A13L33 or GPIIIaPSIHyb-L33). Suitable second GPIIIa polypeptides may include peptides consisting of residues 1 to 450 of SEQ ID NO:1 as described herein (e.g. GPIIIa-ΔβA-L33).

Another aspect of the invention provides a set of polypeptides comprising a first GPIIIa polypeptide and a second GPIIIa polypeptide as described above. For example, a set of polypeptides may comprise a GPIIIa polypeptide having an amino acid sequence which shows at least 80% sequence identity with residues 1-54 of SEQ ID NO:1, a GPIIIa polypeptide having an amino acid sequence which shows at least 80% sequence identity with residues 1-192 of SEQ ID NO:1, a GPIIIa polypeptide having an amino acid sequence which shows at least 80% sequence identity with residues 1-289 of SEQ ID NO:1, a GPIIIa polypeptide having an amino acid sequence which shows at least 80% sequence identity with residues 1-322 of SEQ ID NO:1, a GPIIIa polypeptide having an amino acid sequence which shows at least 80% sequence identity with residues 1-365 of SEQ ID NO:1 and/or a GPIIIa polypeptide having the sequence of SEQ ID NO:1.

A GPIIIa polypeptide that has a proline (P) residue at position 33 may specifically bind to HPA-1b antibodies. A method of detecting HPA-1b antibodies in a sample obtained from an individual may comprise determining the binding of one or more antibodies in the sample to a GPIIIa polypeptide comprising P at residue 33 as described herein, wherein binding to the GPIIIa polypeptide is indicative of the presence of HPA-1b antibodies in the sample.

Methods of detecting HPA antibodies as described above may be carried out in any convenient format. Immunological assays are well-known in the art and many suitable formats are available, for example ELISA, Western blotting, mirror resonance (e.g. Biacore®, (Biacore, Upsala, Sweden), evanescence (DiaMed AG, Cressier sur Morat, Switzerland) or Luminex beads (Luminex® system, Austin, Tex., USA).

In certain exemplary embodiments, a sandwich assay format may be employed.

In some embodiments, a sandwich assay may employ an immobilized GPIIIa polypeptide to bind HPA antibodies in the sample and an antibody-binding molecule to detect the presence of antibodies which are bound to the polypeptide.

In other embodiments, a sandwich assay may employ an immobilized antibody-binding molecule to bind antibodies in the sample and a GPIIIa polypeptide as described herein to detect the presence of HPA antibodies bound to the immobilized antibody-binding molecule.

In other embodiments, a competition assay may employ an recombinant HPA-1a antibody-binding molecule (e.g. CamTran007 or any other suitable recombinant antibody with HPA-1a specificity) to bind the GPIIIa polypeptide with L at position 33 as described herein and the inhibition of binding of the GPIIIa-L33 polypetide to the recombinant HPA-1a antibody by polyclonal HPA-1a antibodies present in a serum, plasma or blood sample.

Suitable antibody-binding molecules for use in such methods may include immunoglobulin-binding antibodies, for example anti-human Ig antibodies, anti-human Ig antibodies, anti-human antibodies specific for Ig isotypes or for subclasses of IgG, Staphylococcal protein A and Staphylococcal protein G.

The non-immobilized antibody-binding molecule or polypeptide may comprise a detectable label as described above. Linkage of detectable labels to an antibody-binding molecule or polypeptide may be direct or indirect, covalent, e.g. via a polypeptide bond, or non-covalent. Linkage via a polypeptide bond may be as a result of recombinant expression of a gene fusion encoding specific binding member (e.g. antibody) and label molecule. Linkage via a non-covalent bond may be a result of a binding between a biotinylated antibody and a streptavidin/avidin linked label molecule.

Labels include fluorochromes such as fluorescein, rhodamine, phycoerythrin, Europium and Texas Red, chromogenic dyes such as diaminobenzidine, radioisotopes, macromolecular colloidal particles or particulate material such as latex beads that are colored, magnetic or paramagnetic, binding agents such as biotin and digoxigenin, and biologically or chemically active agents that can directly or indirectly cause detectable signals to be visually observed, electronically detected or otherwise recorded, for example in a FACS, ELISA, Western blot, TRFIA, immunohistochemistry, evanescence, Luminex bead array, or dipstick or other lateral flow assay format.

Biologically or chemically active agents include enzymes, which catalyze reactions that develop or change colors or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples include horseradish peroxidase and chemiluminescence.

In some embodiments, the non-immobilized antibody-binding molecule or polypeptide may be detected using an antibody which binds to said non-immobilized antibody-binding molecule or polypeptide. A suitable detection antibody may be labelled.

Other aspects of the invention relate to the provision of kits for detecting HPA antibodies.

A GPIIIa polypeptide as described above may form part of a kit for detecting or diagnosing HPA antibodies e.g. in a suitable container such as a vial in which the contents are protected from the external environment.

A kit for detecting an HPA antibody in a sample may comprise a GPIIIa polypeptide as described above, and one or more detection reagents for determining binding of one or more antibodies in a sample to the GPIIIa polypeptide. The GPIIIa polypeptide may be free in solution or may be immobilized on a solid support, such as a magnetic bead, tube or microplate well.

In some embodiments, the kit may further comprise an antibody-binding molecule. Suitable antibody-binding molecules include Staphylococcal protein A, Staphylococcal protein G and antibodies that bind to immunoglobulins, such as IgG, in particular human immunoglobulins.

The antibody-binding molecule may be a capture or detection reagent and may be free in solution or may be immobilized on a solid support, such as a magnetic bead, tube or microplate well.

The antibody-binding molecule or polypeptide may be labelled with a detectable label, for example a fluorescent or chromogenic label or a binding moiety such as biotin. Suitable labels are described in more detail above.

The detection reagents may further comprise a substrate, for example a chromogenic, fluorescent or chemiluminescent substrate, which reacts with the label, or with molecules, such as enzyme conjugates, which bind to the label, to produce a signal. The detection reagents may further comprise buffer solutions, wash solutions, etc.

The kit may also comprise one or both of: apparatus for handling and/or storing the sample obtained from the individual; and, apparatus for obtaining the sample from the individual. This may include, for example, a needle or lancet.

The kit may also include instructions for use of the GPIIIa polypeptide, e.g. in a method of detecting a HPA antibodies in a test sample, as described herein.

Other aspects of the invention relate to the identification of specific binding members and antibody antigen-binding domains which bind to GPIIIa, and may therefore be useful in therapeutic and diagnostic methods HPA-1a and HPA-1b specific recombinant antibodies may be used for HPA-1a and HPA-1b phenotyping of individuals. Antibodies which bind GPIIIa may be used therapeutically, e.g. to prevent binding of fibrinogen or other extracellular matrix proteins to GPIIIa or to ameliorate the binding of harmful viruses to the PSI domain of GPIIIa (e.g. Hanta virus).

A method of producing an antibody which binds GPIIIa may comprise administering an immunogen comprising a GPIIIa polypeptide as described herein to an animal, and isolating from said animal an antibody which binds to said polypeptide. The immunogen may comprise a protein carrier, such as Keyhole Limpet Haemocyanin. Other suitable carriers are well known in the art.

Antibodies may be obtained from immunized animals using any of a variety of techniques known in the art, and screened, typically using binding of antibody to antigen of interest. For instance, Western blotting techniques or immunoprecipitation may be used (Armitage et al. (1992) Nature 357:80-82).

In certain exemplary embodiments, an antibody molecule may be a monoclonal antibody. Methods of producing monoclonal antibodies are well known in the art (see, for example, Harlow et al. Antibodies: A Laboratory Manual, Cold Spring Harbor Laboratory (Cold Spring Harbor, N.Y., 1988) pp. 353-355) and are described in more detail below. For example, antibody-producing cells may be isolated from an immunized mammal and fused with immortalized cells to produce a population of antibody-producing hybridoma cells, which can then be screened to identify a hybridoma cell that produces an antibody which displays optimal binding characteristics.

In some embodiments, a hybridoma may be produced by a method comprising: immunizing a non-human mammal with an immunogen comprising a GPIIIa polypeptide as described above, producing one or more fusions of antibody producing cells from said mammal and immortalized cells to provide a population of hybridoma cells; and screening said population to identify a hybridoma cell which produces an antibody which binds the GPIIIa polypeptide.

In certain exemplary embodiments, the population of hybridoma cells is screened by testing the binding of antibodies produced by cells of the population to one or more GPIIIa polypeptides as described herein. In some embodiments, the relative binding of antibodies to GPIIIa polypeptides with L at position 33 relative to GPIIIa polypeptides with P at position 33 may be determined. Conventional techniques such as western blotting or immunoprecipitation may be used.

Hybridoma cells identified as producing antibodies which bind to the GPIIIa polypeptide may be isolated and/or purified from the population.

Following isolation, the hybridoma may be expanded, maintained and/or cultured in a culture medium using methods which are well-known in the art. Antibodies produced by the hybridoma may be isolated from said culture medium. A method of producing an antibody may comprise culturing a hybridoma cell produced as described above in a culture medium; and isolating from the medium an antibody as described above, for example, an antibody which binds to the GPIIIa polypeptide.

Alternatively, a monoclonal antibody specific for a GPIIIa polypeptide may be obtained from a recombinantly produced library of expressed immunoglobulin variable domains or other molecules comprising antibody antigen-binding domains, e.g. using lambda bacteriophage or filamentous bacteriophage which display functional immunoglobulin binding domains on their surfaces; for instance see WO92/01047. The library may be immunologically naive, that is constructed from sequences obtained from an organism which has not been immunized with a polypeptide comprising the epitope, or may be one constructed using sequences obtained from an organism which has been exposed to the antigen of interest.

A method of producing an anti-GPIIIa binding member comprising contacting a GPIIIa polypeptide as described above with a diverse population of antibody antigen-binding domains, and determining the binding of members of said population to the polypeptide is provided.

The antibody antigen-binding domains may be comprised in antibodies or scFv, Fab, Fv, dAb, Fd or diabody molecules.

An antibody antigen-binding domain may be identified in said population which binds to the GPIIIa polypeptide. In some embodiments, an antibody antigen-binding domain may be identified in said population which binds to GPIIIa polypeptides comprising leucine at position 33 but does not bind to GPIIIa polypeptides comprising proline at position 33 or which binds to GPIIIa polypeptides comprising proline at position 33 but does not bind to GPIIIa polypeptides comprising leucine at position 33.

Antibody antigen-binding domains may be displayed on the surface of virus particles i.e. the diverse population may be a phage display library.

The virus particle which displays the identified antibody antigen-binding domain may be isolated and/or purified and the nucleic acid encoding the antibody antigen-binding domain obtained from said particle.

The nucleic acid encoding the antibody antigen-binding domain may be sequenced and/or expressed to produce the encoded antibody antigen-binding domain that binds to the GPIIIa polypeptide.

An antibody antigen-binding domain produced as described above may be further tested using routine methodology to determine its specificity e.g. to determine whether it binds specifically to GPIIIa polypeptide with L or P at position 33.

In some embodiments, the binding properties of the antibody antigen-binding domain may be further optimized using standard antibody engineering techniques, including affinity maturation, for example by chain shuffling, and site-specific, random or combinatorial mutagenesis.

An antibody antigen-binding domain which is comprised in an antibody or antibody molecule, for example an scFv, Fab, Fv, dAb, Fd or diabody molecule, may be reformatted, for example into an IgG antibody, using standard techniques for subsequent use, for example in a method of HPA phenotyping.

Antibodies, specific binding members and antibody antigen-binding domains which bind GPIIIa polypeptides as described herein may be useful methods of HPA-1 phenotyping. For example, in some embodiments, a method of HPA-1 typing an individual may comprise contacting a specific binding member comprising an antibody antigen-binding domain which specifically binds to GPIIIa polypeptides comprising leucine at position 33, for example an antibody antigen-binding domain identified as described herewith, with a sample of platelets obtained from an individual; and determining the binding of the specific binding member to platelets in the sample. Binding may be indicative that the individual is an HPA-1a homo- or heterozygote. Absence of binding may be indicative that the individual is HPA-1a negative.

In other embodiments, a method of HPA-1 typing an individual may comprise contacting a specific binding member comprising an antibody antigen-binding domain which specifically binds to GPIIIa polypeptides comprising proline at position 33, for example an antibody antigen-binding domain identified as described herewith, with a sample of platelets obtained from an individual; and determining the binding of the specific binding member to platelets in the sample. Binding may be indicative that the individual is an HPA-1b homo- or heterozygote. Absence of binding may be indicative that the individual is HPA-1b negative. A suitable sample may be any platelet-containing sample, for example a blood sample or a fraction or extract thereof. GPIIIa polypeptides as described herein may also be useful in the treatment of blood to remove harmful HPA-1 antibodies.

A method of removing or depleting HPA-1 antibodies from a sample may comprise contacting a sample with an immobilized GPIIIa polypeptide as described herein, wherein HPA-1 antibodies in said sample bind to the polypeptide; and separating the sample from the polypeptide, such that HPA-1 antibodies are removed from the sample. The sample may be a blood, serum or plasma sample. The sample may, for example, be a blood or plasma sample obtained from an individual with HPA-1a antibodies.

The GPIIIa polypeptide may be immobilized on a column or other solid phase. The blood or plasma sample may be brought in contact with the GPIIIa polypeptide, for example by passing the sample over the solid phase, thereby allowing binding of polyclonal HPA-1a antibodies to the polypeptide, to remove completely or reduce the concentration of HPA-1a antibodies in the blood (apheresis). Apheresis procedures are routinely used in the art to obtain plasma from donors or to remove plasma containing harmful antibodies from a patient. GPIIIa polypeptides as described herein may also be useful in the identification of therapeutic compounds.

A method of identifying a compound useful in the treatment of a GPIIIa mediated condition comprising contacting a GPIIIa polypeptide as described herein with a test compound; and determining binding of the polypeptide with test compound. The presence of binding is indicative that the compound is useful in the treatment of a GPIIIa mediated condition.

GPIIIa mediated conditions include viral infections, such as Hantavirus infection, and atherothrombosis.

A compound may, for example, inhibit the binding of Hantavirus to the PSI domain of GPIIIa or reduce the homotypic interaction between platelets or the heterotypic interaction between platelets and the endothelial cells lining the blood vessel wall, by inhibiting the interaction between glycoprotein IIbIIIa and fibrinogen or other extracellular matrix or blood proteins important to atherothrombosis.

Compounds which may be screened using the methods described herein may be natural or synthetic chemical compounds used in drug screening programs. Extracts of plants, microbes or other organisms which contain several characterized or uncharacterized components may also be used.

Combinatorial library technology provides an efficient way of testing a potentially vast number of different compounds for ability to modulate an interaction. Such libraries and their use are known in the art, for all manner of natural products, small molecules and peptides, among others. The use of peptide libraries may be preferred in certain circumstances.

The amount of test compound or compound which may be added to a method of the invention will normally be determined by trial and error depending upon the type of compound used. Typically, from about 0.001 nM to 1 mM or more of test compound may be used, for example from 0.01 nM to 100 μM, e.g. 0.1 to 50 μM, such as about 10 μM.

A method may comprise identifying the test compound as a compound which binds GPIIIa. Such a compound may, for example be useful in the treatment of a GPIIIa-mediated condition.

A test compound identified using one or more initial screens as having ability to bind a GPIIIa polypeptide may be assessed further using one or more secondary screens. A secondary screen may, for example, involve testing for a biological function such as the inhibition of viral infection and amelioration of symptoms of GPIIIa-mediated conditions in animal models.

The test compound may be isolated and/or purified or alternatively, it may be synthesized using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of a GPIIIa-mediated condition. Methods may thus comprise formulating the test compound in a pharmaceutical composition with a pharmaceutically acceptable excipient, vehicle or carrier for therapeutic application.

Various further aspects and embodiments of the present invention will be apparent to those skilled in the art in view of the present disclosure. All documents mentioned in this specification are incorporated herein by reference in their entirety.

Unless context dictates otherwise, the descriptions and definitions of the features set out above are not limited to any particular aspect or embodiment and apply equally to all aspects and embodiments of the invention.

The term “comprising” as used herein encompasses embodiments which include other components and embodiments which do not include other components. The term may be replaced by the term “consisting of” when referring only to the latter embodiments.

The invention encompasses each and every combination and sub-combination of the features that are described above.

Certain aspects and embodiments of the invention will now be illustrated by way of example and with reference to the figures described above.

Table 1 shows the domain structure of the GPIIIaΔBA polypeptide.

TABLE 1

| | Within | | | |
|---|---|---|---|---|
| Fragment Name | Fragment | Cumulative | % of Total | Total aa |
| PSI | 54 | 54 | 12 | 450 |
| Hybrid 1 | 54 | 108 | 24 | 450 |
| bA ins (res55) | 1 | | | 450 |
| Insert Ser (109A) | 1 | | | 450 |
| bA ins (res 361) | 1 | | | 450 |
| Hybrid 2 | 81 | 192 | 43 | 450 |
| Linker | 97 | 289 | 64 | 450 |
| EGF 1 + 2 | 33 | 322 | 72 | 450 |
| EGF 3 + 4 | 43 | 365 | 81 | 450 |
| Beta TD | 85 | 450 | 100 | 450 |
| | 450 | | | |

Table 2 shows the reactivity of 132 polyclonal NAIT samples with recombinant calmodulin-tagged GPIIIa in ELISA. The samples were divided into four distinct clinical groups: (a) cases resulting in intracranial haemorrhage (ICH), (b) platelet count<20X109/L, (c) platelet count≧20X109/L and (4) cases with a history of antenatal treatment.

TABLE 2

| | Anti- | | | | |
|---|---|---|---|---|---|
| | HPA-1a | PSI | PSIHyb | ΔβA | ΔSDL |
| ICH | 15 | 11 | 12 | 15 | 15 |
| Plt | 49 | 35 | 34 | 47 | 46 |
| (<20 × 10⁹/L) | | | | | |
| Plt | 35 | 24 | 20 | 33 | 27 |
| (≧20 × 10⁹/L) | | | | | |
| Treated | 33 | 23 | 23 | 31 | 29 |
| Total | 132 | 93 | 89 | 126 | 122 |

### Example I

### Materials and Methods

Chemicals

All chemicals used in this study were of analytical purity and unless otherwise stated, were obtained from Sigma (Poole, UK). All restriction enzymes were from New England Biolabs (Hitchin, UK).

Antibodies

Anti-GPIIIa, clone Y2/51¹¹ was from Dako, Cambridge, UK and anti-c-Myc, clone 9E10¹² was from the International Blood Group Reference Laboratory (IBGRL), Bristol, UK. Recombinant human anti-D Fog-B¹³ (FOG in the figures) and IgG1 anti-HPA-1a, clone CamTran007¹⁴ were from IBGRL. Human HPA-1a monoclonal antibodies 19-7{Proulx, 1997 #169} and 23-15{Proulx, 1997 #169} were kindly provided by Dr. R. Lémieux, Hema-Québec, Canada. Human polyclonal sera were from the National Blood Service Platelet Immunology serum archives (Cambridge, UK) and were obtained from patients previously referred for investigation of NAIT, PTP and PR. All HPA-1 polyclonal samples (sera or plasma) were obtained from the Platelet Immunology Reference Laboratory (PIRL) at NBS, Cambridge UK. These anti-HPA-1a's had been referred to the PIRL, Cambridge from the South East of England, including London and covering a population of 16 million with 240 000 births annually. Since, there are currently no routine screening in place in England, all samples were referred by physicians based on clinical suspicion of NAIT.

Cloning of GPIIIaΔSDL-P33 Specific Allele and Site-Directed Mutagenesis

The GPIIIaΔSDL-P33 mutant was generated using the Quick Change XL site-directed mutagenesis kit™ (Stratagene, Amsterdam, The Netherlands) according to manufacturer's instructions and using the mutagenic primers 5′-GAGGTGAGCCCGGAGGCAGGGCCTC-3′ (SEQ ID NO:11) and 3′-GAGGCCCTGCCTCCGGGCTCACCTC-5′ (SEQ ID NO:12). Each 50 μL reaction contained 50 ng DNA, 5 μL of 10× reaction buffer, 125 ng of each oligonucleotide primer, 1 μL dNTP, 3 μL QuickSolution, 2.5 U Pfu Turbo. Amplification was performed over 20 cycles consisting of an initial denaturing period at 95° C. for 1 min, 18 cycles each consisting of 50 s at 95° C., 50 s at 60° C. and 9 min 40 s at 68° C. and a final extension time of 7 min at 68° C. The amplified product was digested with 10 U DpnI for 1 h at 37° C. to degrade the parental plasmid. Electrocompetent E. coli TOP10 (Invitrogen) cells were transformed with 2 μL of the DpnI-treated plasmid. Mutation was confirmed by sequencing as described below. The GPIIIaΔSDL-P33/pEF1-puroHis construct generated by site-directed mutagenesis (SDM), was digested with BamHI to recover a GPIIIa fragment containing the P33 mutation. This fragment was then recloned into BamHI digested pEF1-puroHis plasmid to avoid the possibility of introducing unwanted mutations in the vector backbone from the SDM reaction.

DNA Sequencing

All constructs were confirmed by nucleotide sequencing using dye terminator chemistry following manufacturer's instructions (ABI ready reaction mixture; Applied Biosystems, Warrington, UK) using both internal and plasmid-specific primers. Individual clones were sequenced using both forward and reverse primers in separate reactions to ensure concordant results and analyzed by gel electrophoresis on an Applied Biosystems 3100 DNA sequencer. Sequences were analyzed using the Sequence Navigator™ Software.

Cell Culture

Chinese Hamster Ovary (CHO) cell Lec 3.2.8.1 strain was originally engineered to incorporate 4 mutations such that all the glycoproteins are produced with the N-linked carbohydrates in the Man5 oligomannosyl forms and all O-linked carbohydrates truncated to a single GalNAc¹⁷. The single GalcNac was preserved as it has been documented that in some cases, GalcNac is required for the glycoprotein activity.

The CHO Lec cells were maintained in Dulbecco's Modified Eagle's medium (DMEM) (Invitrogen, Groningen, The Netherlands) supplemented with 10% fetal calf serum (Invitrogen), 0.1 mM non-essential amino acids (Invitrogen), 2 mM L-glutamine (Invitrogen), and 1% vol/vol penicillin and streptomycin (Invitrogen) (complete media).

Transfection of CHO Lec 3.2.8.1 Cells

CHO Lec 3.2.8.1 cells were transfected by electroporation with either plasmid containing the allele-specific recombinant GPIIIaΔSDL gene segments. The cells were resuspended in electroporation buffer (NaH₂PO₄.H₂O, Na₂HPO₄.7H₂O, NaCl, pH 7.2) at a concentration of ˜1×10⁷ cells/mL. For each construct, 800 μL resuspended cells were incubated for 10 min on dry ice with DNA (10 μg, GPIIIaΔSDL in pEF1puro and with C-terminal hexahistidine tag) before electroporation (370 V, 960 μF). Following transfection, the cells were allowed to recover for 15 min, resuspended in 9 mL complete media and seeded in a 10 cm tissue culture plate. They were incubated overnight (O/N) at 37° C. and 5% CO₂. Twenty-four hours post-transfection, the cells were washed once and seeded into the original plate. The following day, they were treated with trypsin/EDTA (Invitrogen) and resuspended in selection media (complete media and puromycin 8 μg/mL) before seeding into a 96-well plate (5×10³ cells/mL; 200 μL/well). Fourteen days post transfection the culture supernatants were assayed for secreted GPIIIa oligopeptide by ELISA.

Production and Detection of Soluble Recombinant Monomeric GPIIIa Oligopeptides

Stable clones were screened for secretion of His-tagged GPIIIaΔSDL by ELISA, using the Ni-NTA HisSorb strips (QIAGEN, Crawley, UK), according to manufacturer's instructions. All incubation steps were carried out at room temperature (RT) on a plate shaker. Briefly, the Ni-NTA strips were coated for 2 h with 200 μL of neat supernatant. The wells were washed four times with Phosphate Buffered Saline (PBS) containing 0.05% Tween20 (PBST) before incubation with the MoAb Y2/51 (1 μg/ml in PBS/0.2% Bovine serum albumin (BSA); 200 μL/well) for 2 h. The wells were then washed with PBST and incubated with horseradish peroxidase (HRP)-conjugated goat anti-mouse immunoglobulin G (1:5000 in PBS/0.2% BSA, Sigma). Immune complexes were detected by the addition of 3,3′,5,5′-tetramethylbenzidine (TMB; Sure Blue, KPL, Guildford, UK) and the reaction was stopped with 2.5 M H₂SO₄. Absorbance was read at 450 nm.

Expression in Drosophila Schneider's S2 Cells

Eight rGPIIIa fragments were designed and cloned in the pMT-CaM vectors for expression in the Drosophila Schneider's S2 cells.

The recombinant GPIIIa fragments expressed were GPIIIaΔSDL (residues 1-690 with residues 160-188 deleted), GPIIIaΔβA (residues 1-109 fused to residues 352 to 690 with a serine residue inserted after residue 109), GPIIIaPSIHyb (residues 1-109 fused to residues 352-433 with a serine residue inserted between residue 109 and 352) and GPIIIaPSIA13 (residues 1-54 with the cysteine residue at position 13 mutated to alanine). Each fragment was expressed with either leucine (L) or proline (P) at position 33.

GPIIIaΔSDL

The longest fragment to be expressed is the GPIIIaΔSDL. This fragment consists of residues 1 to 690 with residues 160-188 deleted. The deleted residues form part of the specificity-determining loop (SDL) region and has been shown to form the interface with the GPIIb subunit¹⁶.

GPIIIaΔSDL was cloned into pMT-CaM as a BsmB1 insert. Since, GPIIIa contains most of the restriction sites present in the multiple cloning site of the pMT-CaM vector amongst which Nco1 and Not1, the fragment could not be cloned directly into the vector. Instead, the BsmB1 restriction sites were appended at both ends of the PCR product as this enzyme recognizes a non-palindromic site and cuts outside this region. Thus, by designing primers such that digestion of the PCR products will generate compatible overhangs it was possible to clone GPIIIaΔSDL into the pMT-CaM. GPIIIaΔSDL was amplified by PCR using the primer pairs 5′-TTTCGTCTCACATGGCCGGGCCCAACATCTGTACC (SEQ ID NO:13) (GPIIIaBsmB1F) and 5′-TTTCGTCTCTGGCCGCGGGCCCTTGGGACACTCTGGC (SEQ ID NO:14) (GPIIIaΔSDLBsmB1R) and the GPIIIaΔSDL insert in pEF1-His-puro as template. Each 50 μL PCR reaction contained typically 50 ng DNA, 5 μL of 10× reaction buffer, 125 ng of each primer, 2 mM dNTP, MgCl₂ and 2.5 U Amplitag Gold (Promega). Amplification was performed over 30 cycles consisting of an initial denaturing period at 95° C. for 5 min, 30 cycles each consisting of 1 min at 94° C., 30 s at 60° C. and 1 min 30 s at 72° C. The final extension time was 7 min at 72° C.

GPIIIΔβA

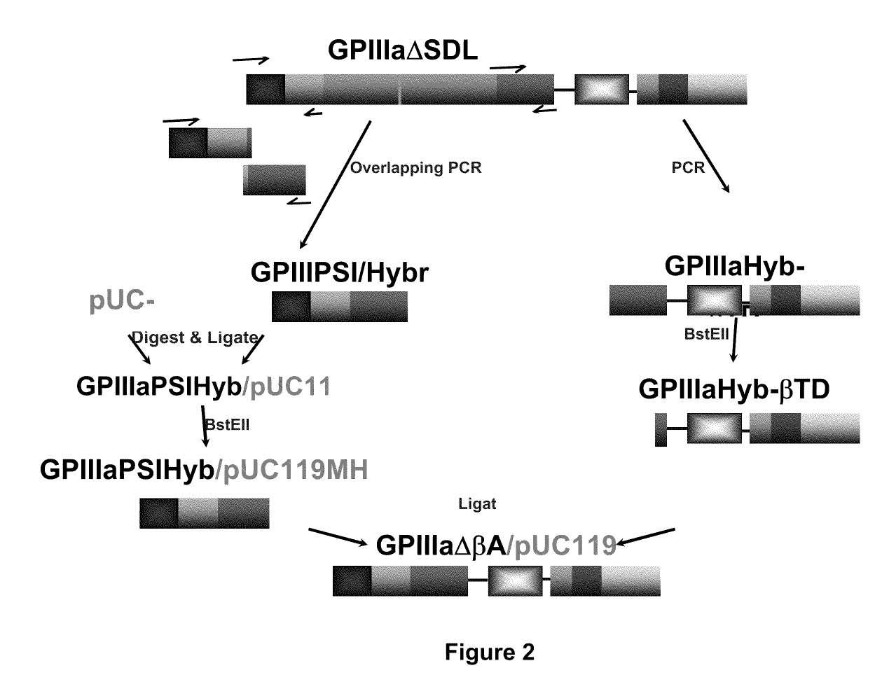

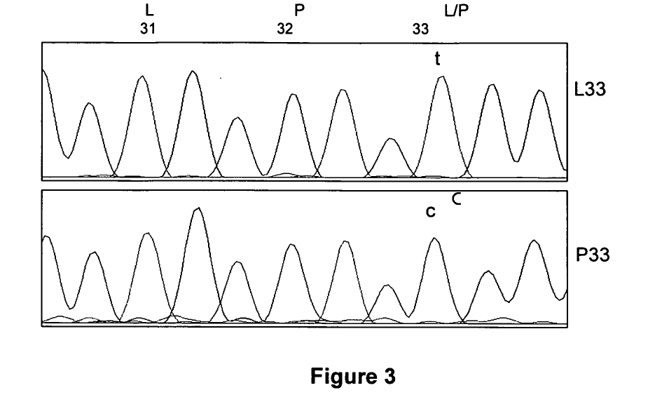

To investigate the role of the PA domain in the HPA-1 epitopes, both allelic forms of GPIIIΔβA constructs were generated. The PSIHyb fusion construct was cloned into the pUC119-MycHis vector, which contains residues 1-438 with the ΔβA deleted, was digested with BstEII, a restriction enzyme which cuts in the PSIHyb insert at nucleotide position 597 (amino acid position 199). Another GPIIIa fragment called GPIIIaHybβTD consisting of residues encoding residues 428 to 690 was also amplified by PCR using GPIIIaΔSDL as template and primers GPIIIaHyb and GPIIIaΔSDLBsmB1R. The GPIIIaHybβTD PCR product was purified using the QIAquick gel purification kit according to manufacturer's instructions was then digested with BsmB1 and BstEII O/N at 55° C. before gel purification. The digested BstEII/BsmB1 GPIIIaHybβTD was then ligated into GPIIIaPSIHyb/puC119MycHis previously digested with BstEII and BsmB1. FIG. 2 is a schematic diagram illustrating the various cloning steps leading to PSIHyb and GPIIIaΔβA.

GPIIIa PSI Hybrid Fusion Gene

GPIIIa gene fusions (L33 and P33), containing the PSI fused to the hybrid domain, were generated by overlapping polymerase chain reaction (PCR). GPIIIa gene fragments encoding residues 1-109 (both the L33 and P33 forms) plus an additional codon for a serine (named residue 109A) at the 3′-end were amplified by PCR using the primers 5′-AACTGCGGCCCAGCCGGCCATGGCCGGGCCCAACATCTGTACCACGCGA (SEQ ID NO:15) (GPIIIa-PSIF) and 5′-AGAACGAGAATCCTCCACCTGCCGCACTTGGATGGAGAA (SEQ ID NO:16). Another gene fragment encompassing residues 352-437 of GPIIIa was amplified using the primers:
5′-CGGCAGGTGGAGGATTCTCGTTCTAAAGTAGAGCTGGAGGTG (SEQ ID NO:17) and primers
5′-ACTTGCGGCCGCGCCTGGCAGGCACAGTCACAATC (SEQ ID NO:18) (GPIIIa-HybridR).

Each 50 μL PCR reaction contained typically 50 ng DNA, 5 μL of 10× reaction buffer, 125 ng of each primer, 2 mM dNTP, MgCl₂ and 2.5 U Amplitag Gold (Promega). Amplification was performed over 30 cycles consisting of an initial denaturing period at 95° C. for 5 min, 30 cycles each consisting of 1 min at 94° C., 30 s at 60° C. and 1 min 30 s at 72° C. The final extension time was 7 min at 72° C.

The PCR products were separated by electrophoresis and the purified products were used as templates to generate the fusion fragments by overlapping PCR using the primers GPIIIa-PSIF and GPIIIa-HybridR. Reactions were out carried as described above and the products were gel-purified. The purified PSIHyb-L33 and PSIHyb-P33 products were digested with NcoI and NotI before ligation into the bacterial expression vector pUC119-MycHis to check for recombinant GPIIIaPSIHyb expression and reactivity with anti-HPA-1a and anti-HPA1b polyclonal antibodies. The PSIHyb inserts L33 and P33 were then cloned into pMT-CaM as Nco1/Not1 inserts.

GPIIIa PSI

The PSI domain consists of residues 1 to 54 inclusive and contains an odd number of cysteine residues namely 7. Cys13 forms a long-range disulphide bond with Cys435¹⁸. To express the PSI domain only and to ensure that the correct disulphide bridges are formed, the cysteine residue at position 13 was mutated to alanine. This was carried out by amplifying a segment of the GPIIIa gene consisting of residues 8 to 438 using the primer pairs 5′-CGAGGTGTGAGCTCCGCCCAGCAGTGCCTGGC (SEQ ID NO:19) (GPIIIaAla13F), which binds at nucleotide 22 of GPIIIa and codes for Ala instead of Cys at residue 13, and primer 5′-ACTTGCGGCCGCGGCCTGGCAGGCACAGTCACAATC (SEQ ID NO:20) (HybNot1R). Following the Cys13Ala mutation, the first few GPIIIa residues and the restriction sites Nco1 and Not1 were appended by amplifying the PSI domain by PCR using the primer pairs 5′-AACTGCGGCCCAGCCGGCCATGGCCGGGCCCAACATCTGTACCACGCGA (SEQ ID NO:21) (PSI Nco1F) and 5′-ACTTGCGGCCGCGATGGATTCTGGGGCACAGTTATCCTT (SEQ ID NO:22) (PSINot1R). Both PSIA13L33 and PSIA13 P33 were generated, sequenced using the plasmid specific primers pMTF and pMTCamR and were then cloned as Nco1/Not1 inserts in pMT-CaM vector.

Drosophila Expression Vector pMT/Bip/Calmodulin

Recombinant protein was expressed in Schneider's Drosophila S2 cells using the Invitrogen's Drosophila Expression System (DES®), which enables recombinant protein expression from both stable mammalian and insect expression systems. This expression system is non-lytic and uses simple plasmids for stable expression. Protein expression occurs in healthy, logarithmically growing cells such that high levels and high-quality recombinant protein can be produced.

The gene of interest is sub-cloned into the pMT/Bip/Calmodulin expression vector (a modified version of the Invitrogen vector pMT/Bip/V5-His DES® in which the V5His tag has been replaced by calmodulin) and co-transfected into S2 cells using the pCoBlast selection vector for stable and inducible expression of secreted protein. The pMT/BiP/V5-His DES® vector contains the Drosophila metallothionein (MT) promoter¹⁹ which allows the inducible expression of the gene of interest. This promoter is tightly regulated, but is easily induced by the addition of copper sulphate (CuSO₄) to the culture medium. The Drosophila BiP gene, which encodes an immunoglobulin-binding chaperone protein leads to the channeling of BiP into the S2 secretory pathway and directs the recombinant protein into the culture medium²⁰.

Maintenance of S2 Cells

Drosophila Melanogaster Schneider's S2 cells (Invitrogen)²¹ were maintained at 2×10⁶ cells/ml, in DES™ Expression Media (Invitrogen) supplemented with 10% heat-inactivated fetal calf serum, 100 units/ml penicillin, 100 units/ml streptomycin, at 25° C. with air as the gas phase. Cells were generally maintained at a density in the range of 6 to 15×10⁶ cells/mL and were seeded at a minimum density of 2×10⁶ cells/mL.

Transfection and Selection

Drosophila Schneider's S2 cells were transfected using the Calcium Phosphate transfection kit (Invitrogen) according to manufacturer's instructions. S2 cells were transfected with DNA-calcium phosphate coprecipitates using the pCoBlast plasmid as the selectable marker to confer blasticidin resistance²². The cells were washed the following day to remove the calcium phosphate solution and 4 days post-transfection, blasticidin (12.5 μg, Sigma) was added to select for stable cells. The cells were washed every 4^{th} day to remove dead cells and replated into the same wells with blasticidin containing media to expand blasticidin-resistant cells. Secreted recombinant proteins were assayed by Western blotting.

Calmodulin Tag Detection Reagent

The calmodulin tag was detected by a calmodulin-binding peptide, here referred to as N9A²³. BSA-conjugated N9A was prepared by the method of Bernatowicz and Matsueda (1986)²⁴. Following conjugation, the BSA-N9A was dialyzed into 100 mM NaCl, 50 mM Na-Borate at pH 8.3 and stored at −20° C., at a concentration of 19 mg/ml as determined by BCA assay (Perbio, Chester, UK). HRP-conjugated-N9A was prepared by mixing N9A peptide with polymerized, maleimide-activated peroxidase (Sigma: P-1834) dissolved in PBS, pH 7.4. This was followed by the addition of 1 mg peroxidase to 4.5 nmoles peptide and the reaction was incubated overnight at 4° C. The free maleimide groups were blocked with 2-mercaptoethanol. HRP-conjugated-9NA was purified using a desalting column and eluted in PBS, pH 7.4. NDSB-201 (0.5 M final; Calbiochem-Novabiochem; Nottingham, UK) and thimerosal (0.02% final; Sigma) was then added to the HRP-conjugated-9NA and the resulting mixture was stored at −20° C. until required.

Detection of CaM-Tagged Proteins by Western Blotting

CaM-Tagged rGPIIIa contained in the S2 supernatant was detected by Western blotting using a semi-dry transblot apparatus (Atto). Following electrophoresis, proteins were transferred onto nitrocellulose membrane (Protran, Schleicher and Schuell, Dassel, USA). The membrane was first soaked with transfer buffer (5.8 gl⁻¹ Tris-base, 2.9 gl⁻¹ glycine, 0.37 gl⁻¹ SDS, 200 ml/L methanol). Proteins were then electro-blotted onto the nitrocellulose membrane for ˜75 mn at 0.1 mA/cm². The membrane was then blocked in 1% BSA in TBST (25 mM Tris HCl (pH 7.4), 0.15 M NaCl, 0.1% Tween) for 2 hours at room temperature. After equilibration, the membrane was incubated with the peroxidase-conjugated 9NA peptide (diluted 1:3 000 in TBST supplemented with 0.5 mM CaCl₂), for 20 min at RT with agitation. Following incubation, the membrane was washed in TBST 3 times for 10 min. Bound antibody-peroxidase conjugate was visualized using ECL detection reagents (Amersham Pharmacia Biotech, UK) according to manufacturer's instructions.

Purification of CaM-Tagged rGPIIIa Proteins

Stably transfected S2 cells were passaged into Ex-Cell 420 (JRH) serum free media (200 ml in 500 ml Erlenmeyer flasks) at a density of 3-4×10⁶ cells/ml and incubated overnight at 26° C. in an orbital shaker (110 rpm). Protein expression was induced by the addition of 500 μM copper sulphate and the cells were grown for up to a further 7 days, following which the supernatant was harvested. The supernatant was cleared by centrifugation at 10 000 rpm for 20 minutes at 4° C. and then passed through a 2 μM filter. CaM-tagged protein was purified from the clarified supernatant on an N-(6-Aminohexyl)-5-chloro-1-naphthalenesulfonamide hydrochloride (W-7)-agarose column.

Twenty milligrams of W-7 was dissolved at RT in 10 ml ethanol. The dissolved W-7 was then diluted in an equal volume of 50 mM sodium borate, pH 8.3 prior to being recirculated for 3 hours on a 1 ml NHS-activated HiTrap column (Amersham Pharmacia Biotech) prepared according to manufacturer's instructions. All purification steps were carried out at 4° C. A flow rate of 0.5 ml/min was used for column preparation and this was increased to 1-1.5 ml/min for protein purification. The W7-agarose column was washed with 20 ml of equilibration buffer (0.5 M NaCl, 50 mM Tris, 1 mM CaCl₂, pH 7.4), 10 ml of elution buffer (0.15 M NaCl, 50 mM Tris, 10 mM EDTA, pH 7.4) and then a further 10 ml of equilibration buffer. The clarified culture supernatants, were supplemented with 0.3 M NaCl, 50 mM Tris, 1 mM CaCl₂, filtered though a 2 uM filter before loading onto the W7-agarose column. Following protein loading, the W7 column was washed with 20 ml equilibration buffer and recombinant CaM-tagged proteins eluted with elution buffer. One ml fractions were collected and analyzed by SDS-PAGE.

Characterization of Recombinantly Expressed CaM-Tagged GPIIIa

The reactivity of five purified recombinant CaM-tagged GPIIIa proteins: PSI-L33, PSIHyb-L33, ΔβA-L33, aΔSDL-L33 and the proline control ΔSDL-P33 (and the two Ig folds of glycoprotein VI [D1D2] as a negative control) with 18 human samples of serum or plasma containing polyclonal HPA-1a antibodies was investigated by ELISA. Recombinant human anti-HPA-1a CamTran007 was used as a positive control.

The wells of a 96 well Maxisorp Plate (Nunc) were coated with 50 μl BSA-9NA (5 μg/ml in 0.05 M Borate buffer) overnight at 4° C. The wells were washed once with 200 μl Wash buffer TBST (50 mM Tris, pH 7.4, 150 mM NaCl, 0.1% Tween, 0.1 mM CaCl₂) before blocking with 10% BSA in Wash buffer for 2 hours at 37° C. Following another 3 washes, the wells were incubated with saturating concentrations of CaM-tagged protein (diluted in TBST supplemented with 1 mM Ca²⁺) for 30 min at RT. Excess CaM-tagged protein was removed by washing the wells 3 times with 200 μl Wash buffer. After a further 3 washes with TBST/Ca²⁻, the wells were incubated with a 1 in 20 diluted human serum/plasma sample for 1 h at RT. Following incubation, the wells were washed 5 times with TBST/Ca²⁺ and bound IgG antibodies were detected with HRP-conjugated goat-anti-human IgG (1:10,000 in TBST/BSA; Jackson, Cambridge, UK). Wells were then washed 6 times with TBST/Ca²⁺. Antibody-HRP complexes were detected by the addition of 3,3′,5,5′-tetramethylbenzidine (TMB; Sure Blue, KPL, Guildford, UK) and the reaction was stopped with 2.5 M H₂SO₄ after 2 minutes of development. Absorbance was read at 450 nm.

GPIIIa Mouse Monoclonal Antibodies and Human V Gene Phage Antibodies

The specificity of the CaM-tagged domain deleted GPIIIa mutants was investigated by ELISA. The GPIIIa specific murine MoAbs CRC54 (Yorkshire Biosciences Ltd, York, UK) (Mazurov, Khaspekova et al. 1996), Y2/51 (Dako, Cambridge, UK) (Gatter, Cordell et al. 1988), SZ21 (Xi, Zhao et al. 1992; Weiss, Goldschmidt-Clermont et al. 1995) and the murine GPIIbIIIa complex specific MoAb RFGP56 (Goodall 1991) was screened for reactivity. As a negative control recombinant GPVI, consisting of the two immunoglobulin folds [D1D2] was included. Fusion proteins were captured via the calmodulin tag in the presence of Ca²⁺ and binding was detected as described previously (Smethurst et al., 2004; O'Connor et al., 2005). Monoclonal murine antibodies were used at 1 μg/ml and 10 μg/ml and detected with HRP conjugated goat-anti-mouse (1:1000 in TBST/Ca²⁺).

Similarly, three HPA-1a specific human monoclonal antibodies 23-15 and 19-7 (both kindly provided by Dr. R. Lémieux, Hema-Quebec, Canada) (Proulx, Chartrand et al. 1997) and the IgG1 anti-HPA-1a clone CamTran007 (International Blood Group Reference Laboratory, Bristol, UK) (Griffin and Ouwehand 1995), were screened. Bound antibodies were detected with HRP-conjugated goat-anti-human (1:10 000 in TBST/BSA; Jackson, Cambridge, UK).

Determination of HPA-1 ELISA Cut-Off

The cut-off for the HPA-1 antibody detection assay was determined by screening 40 anonymous male plasma samples (diluted 1:20 and 1:40, Wellcome Trust Case Control Consortium study, National Blood Service, Cambridge, UK) for reactivity with rGPIIIaΔSDL and rGPIIIaΔβA.

Human HPA-1 Polyclonal Antibodies

To investigate whether the proposed ELISA will detect HPA-1a polyclonal antibodies and how it compares to the current gold standard assay, the MAIPA, binding of both the World Health Organisation (WHO) anti-HPA-1a minimum potency reagent (NIBSC 93/710, National Institute for Biological Standards and Control-NIBSC; Potters Bar, UK) and the WHO proposed anti-HPA-1a standard (NIBSC 03/152, 100 AU) with ΔSDL-L33 and ΔSDL-P33 were investigated. Serial dilutions (1:20 followed by 1:2) of both standards were made and binding to CaM-tagged ASDL was detected as above.

Epitope Mapping of HPA-1a and HPA-1b Human Polyclonal Samples

Human polyclonal HPA-1 samples (132 HPA-1a and 6 HPA-1b) were obtained from the Platelet Immunology Reference Laboratory (PIRL) at NBS, Cambridge UK. The HPA-1a antibodies had been referred to the PIRL, Cambridge and were all positive by MAIPA. The HPA-1a positive samples (diluted 1:20) were screened for reactivity against all four GPIIIa-Leu33 fragments and the GPIIIaΔSDLPro33 fragments by ELISA. Similarly, 5 HPA-1b positive samples from platelet refractory cases were screened for reactivity with all four GPIIIa-Pro33 fragments and the GPIIIaΔSDL-Leu33 fragment.

Results

Selection of Positive GPIIIaΔSDL Transformants

GPIIIaΔSDL-P33 was generated by site-directed mutagenesis from the GPIIIaΔSDL-L33/pEF-His-puro plasmid. After transfection of CHO Lec 3.2.8.1 cells with the pEF1-His-puro plasmids, cells were selected for puromycin resistance over a period of 14 days to isolate stable transfectants. The puromycin-resistant cells were screened for expression of GPIIIaΔSDL by ELISA with the GPIIIa specific mouse MoAbY2/51 and positive clones were expanded and their protein expression level analyzed.

Secretion of GPIIIaΔSDL

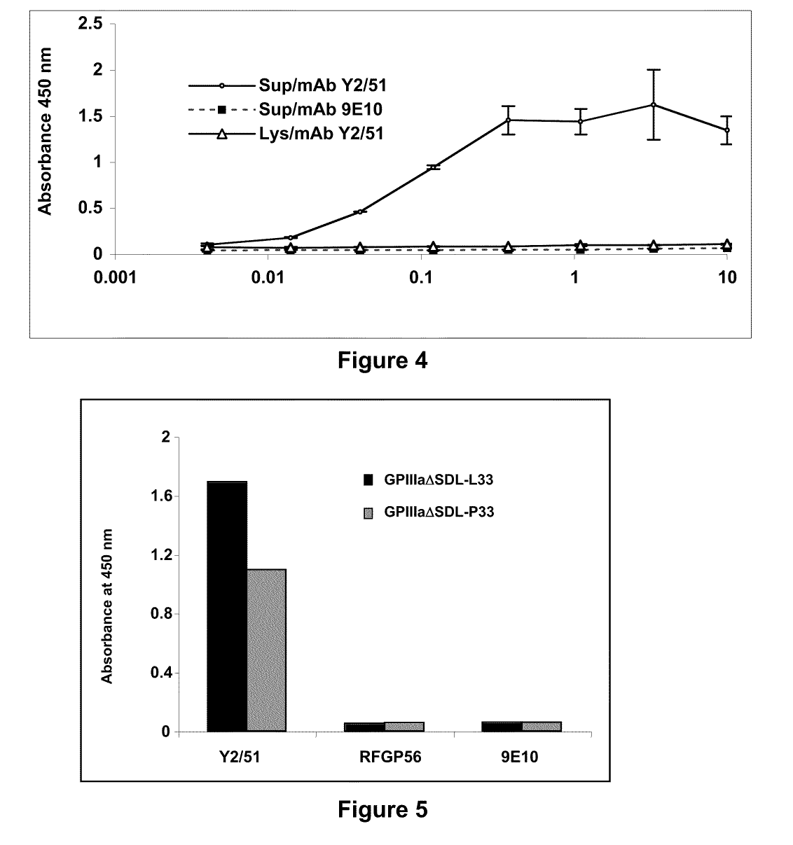

To test whether GPIIIaΔSDL was being secreted by the CHO Lec cells, His-tagged proteins contained in 200 μl of supernatant or cell lysate were immobilized in the wells of ‘Ni-NTA’ coated ELISA plates. Following washing of unbound proteins, the wells were incubated for 1 h with MoAb Y2/51 (0.005-10 μg/mL), a murine antibody against a GPIIIa epitope. The result of the binding of the MoAb Y2/51 at different concentrations to the captured His-tagged protein is shown in FIG. 4. From this data, it was concluded that GPIIIaΔSDL was present in the supernatant but not in the lysate. As expected, no signal was obtained with the negative control MoAb 9E10. Attempts to purify the GPIIIaΔSDL proteins from the CHO Lec supernatant by affinity chromatography were unsuccessful. Hence, further characterization assays were carried out using recombinant GPIIIaΔSDL captured from neat supernatants.

Reactivity of Monoclonal HPA-1a Antibodies with GPIIIaΔSDL

The two allelic forms of GPIIIaΔSDL (133 and P33) were coated on the wells of microtitre plates and tested for reactivity with the murine MoAbs Y2/51 and RFGP56, the latter being an antibody against a compound GPIIbIIIa epitope. Monoclonal antibody Y2/51 was reactive with both allelo-forms of GPIIIaΔSDL whilst, as expected, the negative control MoAbs RFGP56 and 9E10, did not bind (FIG. 5).

Reactivity of Monoclonal and Polyclonal HPA-1 Antibodies with Recombinant GPIIIaΔSDL

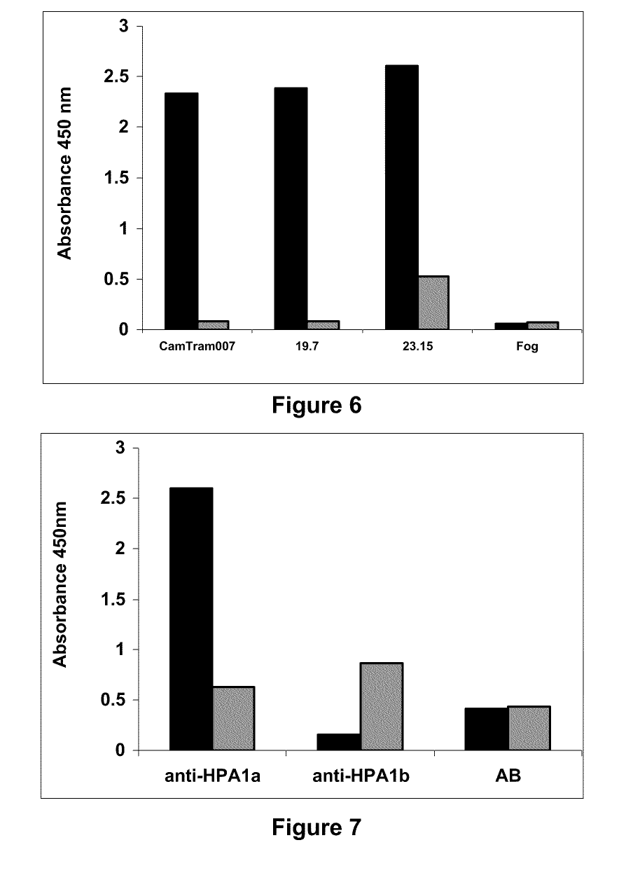

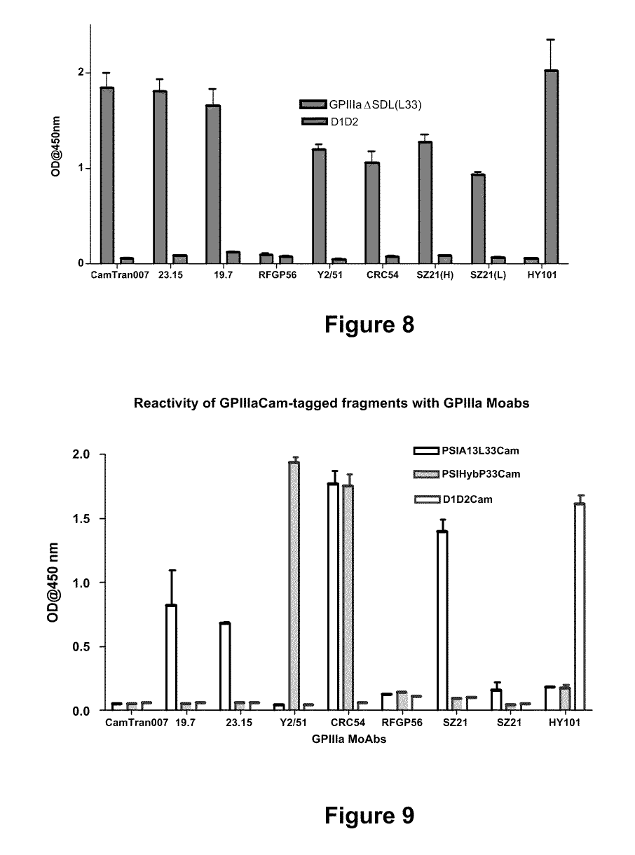

The reactivity of the three human recombinant HPA-1a antibodies, CamTran007, 19.7 and 23.15 with both alleloforms of GPIIIaΔSDL, coated on microtiter plates, was investigated. All three antibodies showed robust reactivity with the GPIIIaΔSDL-L33 form whilst CamTran007 and 19.7 were both negative with GPIIIaΔSDL-P33 (FIG. 6). Interestingly, some reactivity of antibody 23.15 with the proline form of GPIIIaΔSDL was observed. As expected, reactivity of the IgG anti-D Fog-B (Fog in FIG. 6) was negative.

Similarly, two human polyclonal antisera with HPA-1a (anti-HPA-1a) and HPA-1b (anti-HPA-1b) antibodies and a negative control serum (AB) were used in further ELISA experiments with the recombinant GPIIIaΔSDL fragments. The HPA-1a antiserum was from a NAIT referral, the HPA-1b antiserum from a PTP patient and the inert control from a non-transfused group AB male donor. The reactivity of both immune sera with the recombinant GPIIIaΔSDL fragments was as expected; anti-HPA-1a bound GPIIIaΔSDL-L33 and anti-HPA-1b bound GPIIIaΔSDL-P33 (FIG. 7).

Cloning and Expression of rGPIIIa in Drosophila S2 Expression pMT-CaM

Having validated that recombinant GPIIIaΔSDL-L33 expressed in mammalian cells carries the HPA-1a epitope, 8 GPIIIa recombinant gene fragments were cloned in the Drosophila S2 expression vector pMT and recombinant calmodulin tagged protein expressed in the Drosophila Schneider's S2 cells. Both the PSIA13 and the PSIHyb forms were cloned as Nco1/Not1 fragments. GPIIIaΔβA and GPIIIaΔSDL were cloned as BsmB1 fragments into Nco1/Not1 digested pMT-CaM as both Nco1 and Not1 restriction sites are contained with the GPIIIa gene insert. To minimize any constraint between the first and second part of the hybrid domain in both PSIHyb and ΔβA, a serine residue (residue 109A) was inserted at the carboxy-terminus of residue 109 to fuse the β-pleated sheets of the two parts of the hybrid domain. In addition to the removal of the βA domain, the protein was truncated at residue 437 leaving the long-range disulfide bridge between cysteine 13 in the PSI domain and cysteine 435 intact.

Expression of rGPIIIa in Drosophila S2 Cells

All the rGPIIIa gene fragments ligated into pMT-CaM and the plasmids were used to transfect S2 cells. Stable cells were selected over a period of 2-3 weeks. Protein expression was induced by the addition of 500 μM CuSO₄ for 6-7 days. Levels of expressed proteins were assayed by Western blotting using HRP-conjugated-BSA-9NA against CaM. All the 8 rGPIIIa fragments were successfully expressed as CaM-tagged proteins.

Purification of CaM-Tagged rGPIIIa Proteins

Cam-tagged rGPIIIa proteins were purified using a NHS-activated column and several 1 ml fractions were collected and analyzed by SDS-PAGE and typically, rGPIIIa was eluted in fractions 3 to 5. Protein quantification by BCA assay confirmed yields of 10 mg/L for PSIA13-L33, 15 mg/L for PSIHyb-P33 and 14 mg/L for GPIIIaΔβA-L33, 14 mg/L for GPIIIaΔSDL-L33 and 5 mg/L for GPIIIaΔSDL-P33.

Reactivity of Polyclonal Anti-HPA-1a Antibodies with Recombinant CaM-Tagged GPIIIa

The recombinant CaM-tagged GPIIIa fragments were screened for reactivity with a panel of 18 human polyclonal HPA-1a samples from NAIT referrals using the calmodulin-based ELISA. All the 18 samples did react with the two longest fragments (ΔSDL and DβA), albeit reactions were weak for some. Five samples also showed robust reactivity with the two smallest fragments (PSI and PsiHyb). The reactivity with the control antigen D1D2 and with GPIIIaΔSDL-proline 33 was negligible, indicating the interactions between the HPA-1a antibodies and the GPIIIa fragments were allele-specific.

Reactivity with GPIIIa Specific Mouse Monoclonal Antibodies

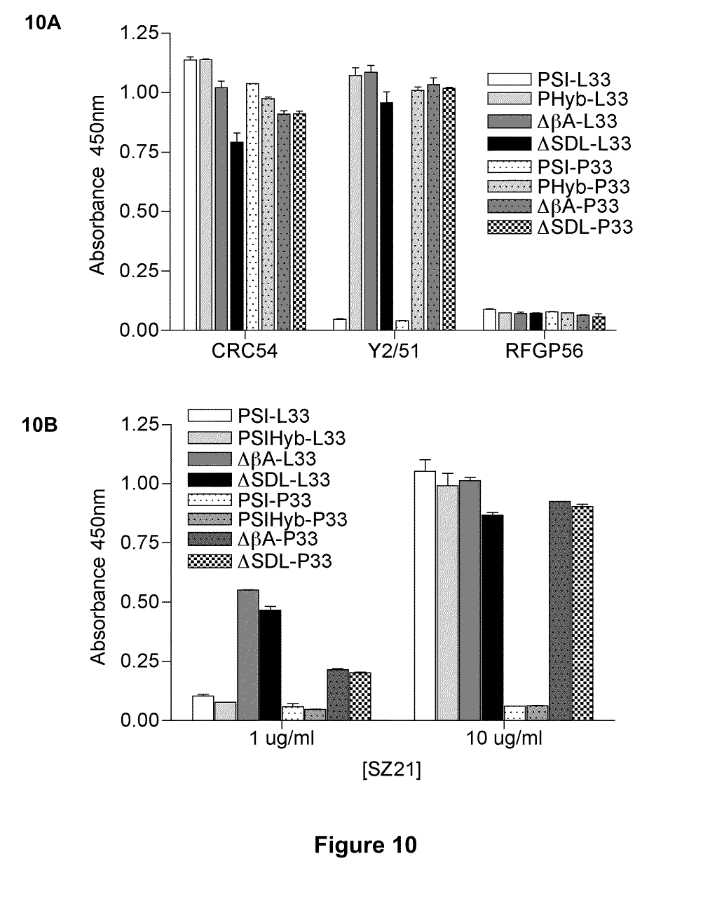

The two sets of four fragments were immobilized on the wells of microtitre plates by targeted binding to BSA derivatized with N9A peptide and the binding of three murine GPIIIa-specific monoclonal antibodies CRC54, Y2/51 and SZ21 was determined (FIG. 10). Clone CRC54 showed robust binding to all 8 fragments and binding was not influenced by the Leu-Pro mutation. Clone Y2/51 is known to bind to a linear amino-terminal GPIIIa oligopeptide of unknown location and here bound to the two sets of the three longest fragments, but was inert against the PSI domain. As expected SZ21 binding to the Leu form was stronger than to the Pro form and at a concentration of 10 μg/mL binding to all 4 Leu-33 fragments was observed while there was minimal reactivity with the two shortest Pro33 fragments. As expected, none of the eight fragments reacted with the control antibodies, anti-GPIIbIIIa RFGP56.

Reactivity with Human V Gene Phage Antibodies Against HPA-1a

Three unique V gene phage antibodies specific for HPA-1a and with similar nanomolar affinities were used to investigate the HPA-1a epitope on the four Leu33 containing GPIIIa fragments. One of the three antibodies CamTran007 (Griffin and Ouwehand 1995) was derived from the B-cells of a HPA-1a immunized mother due to NAIT while both 19-7 and 23-15 (Proulx, Chartrand et al. 1997) were derived form the same V gene phage display library derived form the B-cells of a patient with post transfusion purpura (PTP). Recent studies from our laboratory have shown that the latter two also are reactive with the HPA-1c (Valine33) allele whilst the replacement of Leu33 (HPA-1a) with Valine abolishes CamTran007 binding (Santoso, Kroll et al. 2006). A similar split in reactivity was also observed with another low frequency ITGB3 allele which leads to the mutation of residue 93 in the hybrid domain from arginine to glutamine (Watkins, Schaffner-Reckinger et al. 2002).

CamTran007 only reacted with the two longest Leu33 fragments, ΔβA and ΔSDL while the other two HPA-1a antibodies 19-7 and 23-15 also reacted with the two shortest fragments, PSI-Hybrid and PSI.

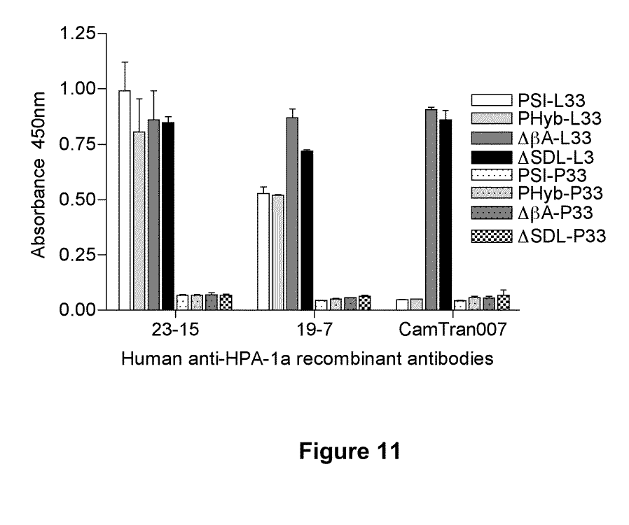

None of the three phage antibodies showed binding to the four Pro33 recombinant proteins, confirming their strict specificity for HPA-1a (FIG. 11).

Reactivity with the WHO Anti-HPA-1a Standards

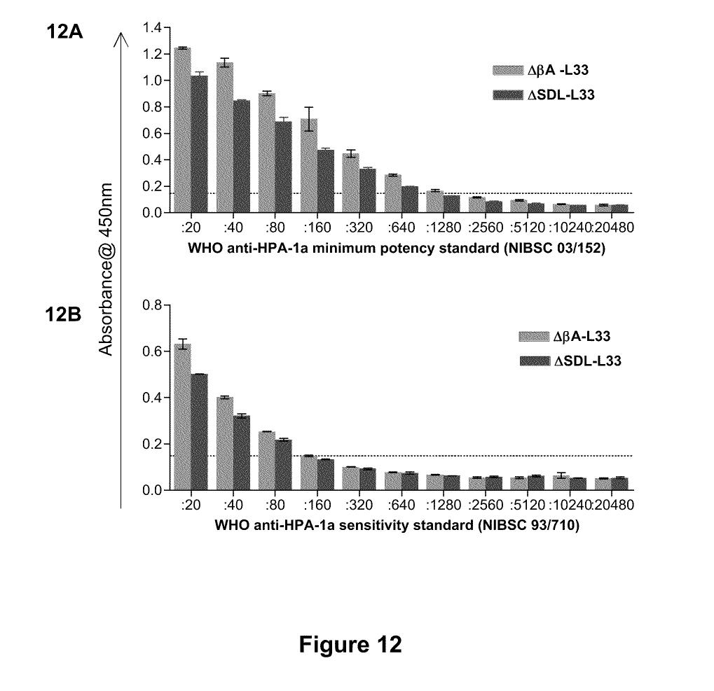

Binding of both the WHO anti-HPA-1a minimum potency reagent (NIBSC 93/710) and the WHO proposed anti-HPA-1a standard (NIBSC 03/152, 100 AU) with ΔSDL-L33 and ΔSDL-P33 were investigated. Both standards could be detected and the ELISA assay was found to be comparable to the MAIPA (FIG. 12).

Reactivity with Polyclonal Human HPA-1a Antibodies from 132 NAIT Referrals

The four GPIIIa-Leu33 and GPIIIa-Pro33-ASDL fragments and GPVI-D1D2 as irrelevant antigen were reacted with the 1 in 20 diluted samples from 132 NAIT cases with HPA-1a antibodies and IgG binding determined by ELISA. All 132 samples contained HPA-1a antibodies as determined by immunofluorescence and MAIPA at the time of referral and were confirmed again by MAIPA during this study.

First, the cut-off of the ELISA assay was determined by testing 40 inert samples. The average and 1×S.D. of the absorbance values was 0.110 and 0.0085 respectively and a cut-off of 0.15 was chosen, resulting in a specificity of 90%. Four of 40 inert samples produced absorbances above the cut-off (0.1585, 0.1675, 0.1995, 0.1855).

The 132 anti-HPA-1a NAIT samples were categorized in four clinical groups: (a) 15 ICH (intracranial haemorrhage) cases with a platelet count<20×109/L, (b) 49 with a platelet count<20×109/L, (c) 35 with a platelet count≧20×109/L and (d) 33 cases with a history of severe HPA-1a immunization and who underwent antenatal treatment. The potency of the samples ranged from 5-193 au/ml and there was no correlation between potency and severity of thrombocytopenia or risk of ICH (Ghevaert et al., 2006). Of the 132 samples tested, 126 were positive with ΔβA-Leu33 (Table 2), including all 15 ICH cases, resulting in a sensitivity of 95%. Six samples with HPA-1a antibodies at the time of referral were negative by ELISA irrespective of the fragment used. However, these six samples were also observed to have low titers when they last tested in the MAIPA. It was interesting to note that four ΔβA-Leu33 positive samples did not react with ASDL-Leu33. Approximately 70% of the 126 samples also showed reactivity with the two shorter Leu33 containing fragments, PSI-hybrid and PSI only. The samples producing positive results with the two longer and the two shorter fragments may contain a mixture of Type I and II antibodies, whilst the 25% of samples only reactive with the two longer fragments contain Type II antibodies only. A detailed analysis of the type of HPA-1a antibodies showed no significant correlations with the clinical categories or the number of pregnancies, e.g. between primi- or multi-parous women. There was no correlation between the strength of reactivity on the two shorter fragments when compared with that on the two longer ones.

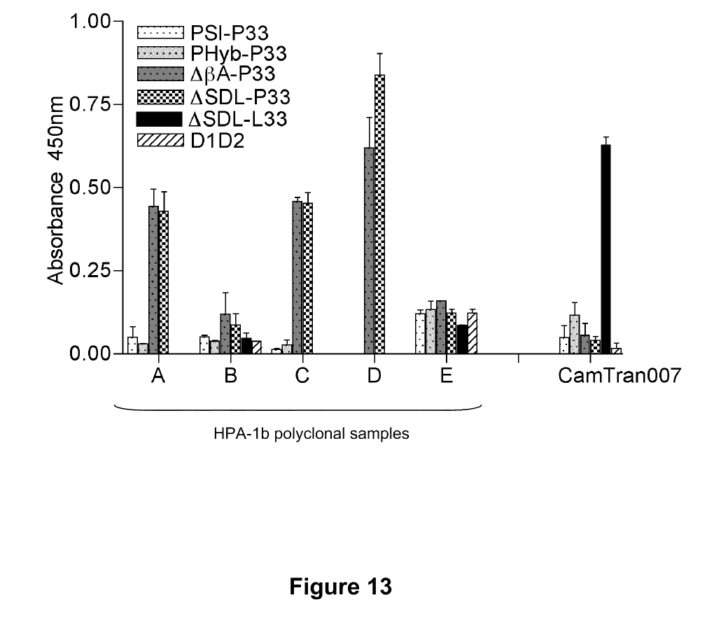

Similarly, to determine whether the recombinant Pro33 containing fragments are suitable for HPA-1b antibody detection, five samples with HPA-1b antibodies all from platelet refractoriness cases were tested. Three of the five samples showed increased IgG binding to the two longest Pro33 fragments, ΔβA and ΔSDL when compared with the corresponding Leu33 fragments (FIG. 13). One of the samples (Sample B) was more positive with ΔβA-Pro and ΔSDL-Pro when compared to the other sample while Sample E was cross-reactive with all the fragments tested. No reactivity with the shorter fragments was observed, an observation compatible with the assumed low affinity of HPA-1b antibodies because of lack of T cell help.

### REFERENCES

1. Mueller-Eckhardt C et al. (1989) Lancet, 1:363-366

2. Newman P J et al. (1989) J. Clin. Invest., 83:1778-1781

3. Goldberger A et al. (1991) Blood, 78:681-687

4. Williamson L M et al. (1998) Blood, 92:2280-2287

5. Ahya R et al. (2001) Trans. and Apheresis Science, 25:139-145

6. Husebekk A et al. (1996) Tidsskr Nor Laegeforen, 116:1219-1222

7. Burrows R F et al. (1993) New Engl. J. Med,. 329:1463-1466

8. Giovangrandi Y et al. (1990) Lancet, 336:310

9. Kunicki T J et al. (1995) Blood Cells Mol. Dis., 21:131-141

10. Goodall A H (1991) Blood Coagul. Fibrinolysis, 2:377-382

11. Gatter K C et al. (1988) Histopathology, 13:257-267

12. Evan G I et al. (1985) Cell, 43:253-261

13. Hughes-Jones N C et al. (1990) Biochem. J., 268:135-140

14. Griffin H M et al. (1995) Blood, 86:4430-4436

15. Proulx C et al. (1997) Vox Sang., 72:52-60

16. Takagi J et al. (2002) Biochemistry, 41:4339-4347

17. Stanley P et al. (19890 Molecular Cell Biology, 9:377-383

18. Xiao T et al. (2004) Nature, 432:59-67

19. Bunch T A et al. (1988) Nucleic Acids Res., 16:1043-1061

20. Kirkpatrick R B et al. (1995) J. Biol. Chem., 270:19800-19805

21. Schneider I. (1972) J. Embryol. Exp. Morphol., 27:353-365

22. Kimura M et al. (1994) Biochim. Biophys. Acta, 1219:653-659

23. Montigiani S et al. (1996) J. Mol. Biol., 258:6-13

24. Bernatowicz M S et al. (1986) Anal. Biochem., 155:95-102

25. Barron-Casella, E. A. et al. (1999) Blood, 93(9) :2959-2967

26. Gatter, K. C. et al. (1988) Histopathology, 13(3):257-267

27. Goodall, A. H. (1991) Blood Coagul. Fibrinolysis, 2(2):377-382

28. Mazurov, A. V. et al. (1996) FEBS Lett., 391(1-2):84-88

29. Newman, P. J. et al. (1989) J. Clin. Invest., 83(5):1778-1781

30. Proulx, C. et al. (1997) Vox Sang., 72(1): 52-60

31. Santoso, S., H. Kroll et al. (2006) Transfusion, 46(5):790-799

32. Valentin, N. et al. (1995) Blood, 85(11) :3028-3033

33. Watkins, N. A. et al. (2002) Blood, 99(5):1833-1839

34. Weiss, E. J. et al. (1995) Tissue Antigens, 46(5):374-381

35. Williamson, L. M. et al. (1998) Blood, 92(7):2280-2287

37. Xi, X., Y. Zhao, et al. (1992) Nouv. Rev. Fr. Hematol., 34(3) :239-242

1. A GPIIIa polypeptide comprising an amino acid sequence which has at least 80% sequence identity to residue 1 to at least residue 54 of SEQ ID NO: 1.

2. (canceled)

3. The polypeptide according to claim 1, comprising an amino acid sequence which has at least 80% sequence identity with a sequence selected from the group consisting of SEQ ID NO: 1, residues 1 to 54 of SEQ ID NO: 1, residues 1 to 108 of SEQ ID NO: 1, residues 1 to 192 of SEQ ID NO: 1, residues 1 to 289 of SEQ ID NO: 1, residues 1 to 322 of SEQ ID NO: 1 and residues 1 to 365 of SEQ ID NO: 1.

4. The polypeptide according to claim 1, wherein the polypeptide has an L at position 33.

5. The polypeptide according to claim 1, wherein the polypeptide has a P at position 33.

6. The polypeptide according to claim 1, wherein the polypeptide includes one or more amino acids selected from the group consisting of a Q at position 62, an A at position 166, a Q at position 248 and a C at position 395.

7. The polypeptide according to claim 1, comprising an amino acid sequence selected from the group consisting of residues 1 to 54 of SEQ ID NO: 1, residues 1 to 108 of SEQ ID NO: 1, residues 1 to 192 of SEQ ID NO: 1, residues 1 to 289 of SEQ ID NO: 1, residues 1 to 322 of SEQ ID NO: 1, residues 1 to 365 of SEQ ID NO: 1 and SEQ ID NO: 1.

8. The polypeptide according to claim 7, comprising an L to P substitution at residue 33.

9-11. (canceled)

12. A nucleic acid encoding a polypeptide according to claim 1.

13. (canceled)

14. A vector comprising a nucleic acid according to claim 12.

15. A host cell comprising a vector according to claim 14.

16. A method of detecting an HPA antibody in a sample obtained from an individual comprising:
determining the binding of one or more antibodies in the sample to a GPIIIa polypeptide comprising an amino acid sequence which has at least 80% sequence identity to residue 1 to at least residue 54 of SEQ ID NO: 1,
wherein the binding of one or more antibodies to the polypeptide is indicative of the presence of an HPA antibody in the sample.

17. (canceled)

18. The method according to claim 16, wherein the HPA antibody is an HPA-1 antibody.

19. The method according to claim 18, wherein the GPIIIa polypeptide comprises L at position 33 and the HPA-1 antibody is an HPA-1a antibody; the GPIIIa polypeptide comprises P at position 33 and the HPA-1 antibody is an HPA-1b antibody; the GPIIIa polypeptide comprises Q at position 62 and the HPA-1 antibody is an HPA-10bw antibody; the GPIIIa polypeptide comprises A at position 166 and the HPA-1 antibody is an HPA-7bw antibody; the GPIIIa polypeptide comprises Q at position 248 and the HPA-1 antibody is an HPA-6bw antibody; or the GPIIIa polypeptide comprises C at position 396 and the HPA-1 antibody is an HPA-8bw antibody.

20-24. (canceled)

25. The method according to claim 16, wherein the HPA antibody is an alloantibody and the binding of the alloantibody with four different GPIIIa polypeptides is determined.

26. The method according to claim 16, comprising:
contacting immobilized GPIIIa polypeptide with the sample; and
determining the binding of antibodies in the sample to the immobilized GPIIIa polypeptide.

27. The method according to claim 16, comprising:
contacting the sample with an immobilized antibody-binding molecule to immobilize antibodies in the sample; and
determining the binding of the immobilized antibodies to the GPIIIa polypeptide.

28. A method of producing an antibody which binds GPIIIa comprising:
administering an immunogen comprising a GPIIIa polypeptide to an animal; and
isolating from said animal an antibody which binds to said polypeptide,
wherein the GPIIIa polypeptide comprises an amino acid sequence which has at least 80% sequence identity to residue 1 to at least residue 54 of SEQ ID NO: 1.

29. A method of producing a hybridoma which produces an antibody which binds GPIIIa comprising:
immunizing a non-human mammal with an immunogen comprising a GPIIIa polypeptide;
producing one or more fusions of antibody producing cells from said mammal and immortalized cells to provide a population of hybridoma cells; and
screening said population to identify a hybridoma cell which produces an antibody which binds the GPIIIa polypeptide,
wherein the GPIIIa polypeptide comprises an amino acid sequence which has at least 80% sequence identity to residue 1 to at least residue 54 of SEQ ID NO: 1.

30. A method of producing an antibody which binds GPIIIa comprising:
culturing a hybridoma cell produced by a method according to claim 29 in a culture medium; and
isolating from the medium an antibody which binds to the GPIIIa polypeptide,
wherein the GPIIIa polypeptide comprises an amino acid sequence which has at least 80% sequence identity to residue 1 to at least residue 54 of SEQ ID NO: 1.

31. A method of producing an antibody which binds GPIIIa comprising:
contacting a GPIIIa polypeptide according to claim 1 with a diverse population of antibody-antigen binding domains; and
determining the binding of members of said population to said GPIIIa polypeptide,
wherein the GPIIIa polypeptide comprises an amino acid sequence which has at least 80% sequence identity to residue 1 to at least residue 54 of SEQ ID NO: 1.

32. The method according to claim 31, wherein the antibody-antigen binding domains are displayed on the surface of virus particles.

33-34. (canceled)

35. A method of removing HPA antibodies from a sample comprising:
contacting a sample with an immobilized GPIIIa polypeptide according to claim 1;
causing or allowing HPA antibodies in said sample bind to the polypeptide; and
separating the sample from the GPIIIa polypeptide, such that the HPA antibodies are removed from the sample.
wherein the GPIIIa polypeptide comprises an amino acid sequence which has at least 80% sequence identity to residue 1 to at least residue 54 of SEQ ID NO: 1.

36. (canceled)

37. A method of identifying a compound useful in the treatment of a GPIIIa-mediated condition comprising:
contacting a GPIIIa polypeptide with a test compound; and
determining binding of the polypeptide with test compound,
wherein the GPIIIa polypeptide comprises an amino acid sequence which has at least 80% sequence identity to residue 1 to at least residue 54 of SEQ ID NO: 1.

38. The method according to claim 37, wherein the GPIIIa-mediated condition is a viral infection or atherothrombosis.

39. A kit for detecting an HPA antibody in a sample comprising:
a GPIIIa polypeptide according to claim 1; and
one or more detection reagents for determining binding of HPA antibodies in a sample to the GPIIIa polypeptide,
wherein the GPIIIa polypeptide comprises an amino acid sequence which has at least 80% sequence identity to residue 1 to at least residue 54 of SEQ ID NO: 1.

40-42. (canceled)


SEQUENCE LISTING (22 sequences)

SEQ ID NO: 1 - protein, 450 aa, Artificial Sequence
  Sequence of GPIIIa deletion construct expressed by recombinant vector
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDSRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDREPYMTENTCNRYCRDEIESVKELKDTGKDAVNCTYKNEDDCVVRFQYYEDSSGKSILYVVEEPECPKGP

SEQ ID NO: 2 - DNA, 1350 bp, Artificial Sequence
  DNA sequence of GPIIIa deletion construct expression vector
gggcccaacatctgtaccacgcgaggtgtgagctcctgccagcagtgcctggctgtgagccccatgtgtgcctggtgctctgatgaggccctgcctctgggctcacctcgctgtgacctgaaggagaatctgctgaaggataactgtgccccagaatccatcgagttcccagtgagtgaggcccgagtactagaggacaggcccctcagcgacaagggctctggagacagctcccaggtcactcaagtcagtccccagaggattgcactccggctccggccagatgattcgaagaatttctccatccaagtgcggcaggtggaggattctcgttctaaagtagagctggaagtgcgtgacctccctgaagagttgtctctatccttcaatgccacctgcctcaacaatgaggtcatccctggcctcaagtcttgtatgggactcaagattggagacacggtgagcttcagcattgaggccaaggtgcgaggctgtccccaggagaaggagaagtcctttaccataaagcccgtgggcttcaaggacagcctgatcgtccaggtcacctttgattgtgactgtgcctgccaggcccaagctgaacctaatagccatcgctgcaacaatggcaatgggacctttgagtgtggggtatgccgttgtgggcctggctggctgggatcccagtgtgagtgctcagaggaggactatcgcccttcccagcaggacgaatgcagcccccgggagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccgggagccctacatgaccgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccct

SEQ ID NO: 3 - protein, 10 aa, Artificial Sequence
  Affinity tag generated by expression vector
MRGSHHHHHH

SEQ ID NO: 4 - protein, 8 aa, Artificial Sequence
  Affinity tag generated by expression vector
DYKDDDDK

SEQ ID NO: 5 - protein, 15 aa, Artificial Sequence
  Affinity tag generated by expression vector
KETAAAKFERQHMDS

SEQ ID NO: 6 - protein, 8 aa, Artificial Sequence
  Affinity tag generated by expression vector
WSHPQFEK

SEQ ID NO: 7 - protein, 10 aa, Artificial Sequence
  Affinity tag generated by expression vector
EQKLISEEDL

SEQ ID NO: 8 - protein, 12 aa, Artificial Sequence
  Affinity tag generated by expression vector
MKAEFRRQESDR

SEQ ID NO: 9 - protein, 12 aa, Artificial Sequence
  Affinity tag generated by expression vector
MRDALDRLDRLA

SEQ ID NO: 10 - protein, 6 aa, Tobacco etch virus
  misc_feature (2)..(2): X is any amino acid
EXXYXX

SEQ ID NO: 11 - DNA, 25 bp, Artificial Sequence
  Mutagenic primer
gaggtgagcccggaggcagggcctc

SEQ ID NO: 12 - DNA, 25 bp, Artificial Sequence
  Mutagenic primer
ctccactcgggcctccgtcccggag

SEQ ID NO: 13 - DNA, 35 bp, Artificial Sequence
  PCR primer
tttcgtctcacatggccgggcccaacatctgtacc

SEQ ID NO: 14 - DNA, 37 bp, Artificial Sequence
  PCR primer
tttcgtctctggccgcgggcccttgggacactctggc

SEQ ID NO: 15 - DNA, 49 bp, Artificial Sequence
  PCR primer
aactgcggcccagccggccatggccgggcccaacatctgtaccacgcga

SEQ ID NO: 16 - DNA, 39 bp, Artificial Sequence
  PCR primer
agaacgagaatcctccacctgccgcacttggatggagaa

SEQ ID NO: 17 - DNA, 42 bp, Artificial Sequence
  PCR primer
cggcaggtggaggattctcgttctaaagtagagctggaggtg

SEQ ID NO: 18 - DNA, 35 bp, Artificial Sequence
  PCR primer
acttgcggccgcgcctggcaggcacagtcacaatc

SEQ ID NO: 19 - DNA, 32 bp, Artificial Sequence
  PCR primer
cgaggtgtgagctccgcccagcagtgcctggc

SEQ ID NO: 20 - DNA, 36 bp, Artificial Sequence
  PCR primer
acttgcggccgcggcctggcaggcacagtcacaatc

SEQ ID NO: 21 - DNA, 49 bp, Artificial Sequence; identical to SEQ ID NO: 15
  PCR primer

SEQ ID NO: 22 - DNA, 39 bp, Artificial Sequence
  PCR primer
acttgcggccgcgatggattctggggcacagttatcctt